(19) United States
(12) Patent Application Publication
(10) Pub. No.: US 2025/0082236 A1
(43) Pub. Date: Mar. 13, 2025

(54) Scalable Manufacturing of Microneedle Arrays Using Automated High-Throughput Manufacturing Systems and High-Capacity Molding

(71) Applicant: Carnegie Mellon University, Pittsburgh, PA (US)

(72) Inventors: O. Burak Ozdoganlar, Sewickley, PA (US); Toygun Cetinkaya, Pittsburgh, PA (US); Ant Yucesoy, Pittsburgh, PA (US); Ali Alp Gurer, Pittsburgh, PA (US)

(21) Appl. No.: 18/580,237

(22) PCT Filed: Jul. 19, 2022
(86) PCT No.: PCT/US22/37610

§ 371 Date: Jan. 18, 2024

Related U.S. Application Data
(60) Provisional application No. 63/223,342, filed on Jul. 19, 2021.

(51) Int. Cl. A61B 5/15

(52) U.S. Cl. CPC A61B 5/150282; A61B 5/150984

(57) ABSTRACT

A manufacturing method for making a plurality of microneedle arrays (MNAs) includes: dispensing a polymer resin into a plurality of wells of at least one mold by an automated dispenser comprising at least one dispensing nozzle; centrifuging the at least one mold to distribute the dispensed polymer resin within the plurality of wells of the at least one mold; curing, solidifying, and/or drying the polymer resin within the plurality of wells of the at least one mold; and removing individual molded MNA parts from the at least one mold with at least one electromechanical mover controlled by at least one computer processor. A mold for making a plurality of the MNAs and a high-throughput manufacturing system for making the MNAs using the mold are also provided.

### CROSS REFERENCE TO RELATED APPLICATION

The present application claims the benefit of U.S. Provisional Patent Appl. No. 63/223,342, filed Jul. 19, 2021, the disclosure of which is hereby incorporated by reference in its entirety.

### BACKGROUND OF THE INVENTION

### Field of the Invention

The present disclosure is directed to manufacturing systems and methods for making microneedle arrays by molding, such as molding using a spin-casting technique, and, in particular, to high-capacity molds and high-throughput manufacturing systems and methods for scalable manufacturing of microneedle arrays in a precise, reproducible, and flexible manner.

### Description of Related Art

To ensure effectiveness and efficiency, doses of vaccines and other therapeutic agents need to be delivered by reliable delivery tools that deliver the vaccines or therapeutic agents to appropriate body tissues in sufficient concentration to produce a desired physiological response. Currently available delivery techniques for delivering vaccines and other flowable therapeutic agents to patients have remained generally unchanged for many years. In particular, prevailing delivery methods often deliver the therapeutic agent via an intramuscular (IM) route using a hypodermic needle. IM injections can be inefficient for creating potent and sustained immunity due to muscle's low immunogenic activity. To circumvent such inefficiencies, a large dose of a vaccine often must be delivered to muscular tissue, which increases both vaccine toxicity and the per-dose cost for the vaccine. The need for large vaccine doses can also contribute to vaccine shortages, especially for vaccines that need to be provided to large patient populations. Needing to use large vaccine doses can also create problems related to cold-chain transportation and storage of vaccine doses. Following an IM injection, used needle sharps must be disposed of in an appropriate manner in compliance with regulations for disposal of biohazardous materials, which further contributes to the complexity and inefficiency of IM injections.

In view of difficulties and inefficiencies related to IM injections, there is a need for different delivery tools for rapid mass vaccination and delivery of other therapeutic agents to patients, which can be used to vaccinate or treat large patient populations during, for example, current, ongoing, or future pandemics, such as the current coronavirus pandemic and/or future pandemics. One alternative option for the delivery of vaccines and other therapeutic agents to patients is using microneedles and microneedle arrays (MNAs), which are also known as microarray patches (MAPs). In some limited examples, MNAs have been used for the delivery of drugs, vaccines, viral vectors, stem cells, nucleic acids (mRNA, microRNA, siRNA, DNA), and other bioactive agents to humans. Currently, MNAs are primarily used for skin-based intradermal and transdermal delivery. MNAs have also been used for delivering drugs to other organs, including buccal and sublingual delivery via the oral cavity, as well as for ocular delivery of therapeutic agents and for delivery of certain therapeutic agents to the brain, heart, or liver.

However, currently available MNAs are often produced using laboratory or small-scale production methods, which would be difficult or impossible to scale up for, e.g., use during a mass vaccination event or for obtaining a large number of therapeutic devices (e.g., for diabetes). Accordingly, there is a need for new methods and systems for producing MNAs, which can be optimized for high-throughput, efficient, and reproducible MNA manufacturing in compliance with accepted manufacturing practices.

### SUMMARY OF THE INVENTION

The systems and methods of the present disclosure are configured to provide automated manufacturing of MNAs at production rates sufficient to support mass vaccination and/or large-scale drug delivery efforts. The manufacturing methods and systems disclosed herein are also intended to be flexible and adaptable so that the disclosed methods and systems can be used to manufacture different types of MNAs, including dissolvable, hybrid, hollow (or thru-hole), and coated MNAs.

According to an aspect of the disclosure, a manufacturing method for making a plurality of microneedle arrays (MNAs) includes: dispensing a polymer resin into a plurality of wells of at least one mold by an automated dispenser comprising at least one dispensing nozzle; centrifuging the at least one mold to distribute the dispensed polymer resin within the plurality of wells of the at least one mold; curing, solidifying, and/or drying the polymer resin within the plurality of wells of the at least one mold; and removing individual molded MNA parts from the at least one mold with at least one electromechanical mover controlled by at least one computer processor.

According to another aspect of the disclosure, a mold for making a plurality of microneedle arrays (MNAs) includes a tray comprising a top surface, a bottom surface, and a sidewall extending between the top surface and the bottom surface. The mold further includes a plurality of wells on the top surface of the tray. Each well includes an open top, a closed bottom, and an inner surface extending between the top and the bottom. The mold further includes a plurality of fiducial markers on the tray that can be identified in images of the mold captured by optical sensors for aligning the mold with a dispenser of an MNA manufacturing system.

According to another aspect of the disclosure, a high-throughput manufacturing system for making microneedle arrays (MNAs) includes: at least one mold for making a plurality of microneedle arrays (MNAs); at least one dispenser that dispenses a polymer resin material for forming a molded MNA part to a plurality of wells of the at least one mold; at least one centrifuge for centrifuging the at least one mold causing the polymer resin material dispensed into the wells of the mold to distribute through the wells; and at least one curing or drying device for exposing the filled at least one mold to heat and/or radiation causing the polymer resin material to cure or dry. The system further includes at least one automated or robotic electromechanical mover for moving the at least one mold through the dispenser, at least one centrifuge, and at least one curing or drying device, for demolding the molded MNA parts from the at least one mold, and for assembling the MNAs from the molded MNA parts.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limit of the invention.

### BRIEF DESCRIPTION OF THE DRAWINGS

FIG. 1A is a perspective view of a microneedle array, according to an example of the present disclosure;

FIG. 1B is a cross-sectional view of the microneedle array of FIG. 1A;

FIG. 2A is a perspective view of a microneedle array mold for molding micro-cannula of the array, according to an example of the present disclosure;

FIG. 2B is an expanded cross-sectional view of a portion of the mold of FIG. 2A;

FIG. 3A is a perspective view of another example of a microneedle array mold for molding microneedle tips of the array, according to an example of the present disclosure;

FIG. 3B is an expanded cross-sectional view of a portion of the mold of FIG. 3A;

FIG. 4A is a perspective view of a manufacturing unit for making microneedle arrays, according to an example of the present disclosure;

FIG. 4B is a drawing of the dispensers of the manufacturing unit of FIG. 4A;

FIG. 4C is a schematic drawing of the manufacturing unit of FIG. 4A;

FIG. 5A is a perspective view of a centrifuge bucket containing a support and microneedle array molds loaded with adapters, according to an example of the present disclosure;

FIG. 5B is a cross-sectional view of the centrifuge bucket of FIG. 5A;

FIG. 5C is a perspective view of the support containing the microneedle array molds of FIG. 5A;

FIG. 6A is a schematic drawing showing steps for making dissolvable tips of a microneedle array, according to an example of the present disclosure;

FIG. 6B is a schematic drawing showing steps for making micro-cannula of a microneedle array, according to an aspect of the present disclosure;

FIG. 6C is a schematic drawing showing steps for assembling a hybrid microneedle array including microneedle cannula with dissolvable microneedle tip, according to an example of the present disclosure; and

FIGS. 7A and 7B are flow charts showing steps for making microneedle arrays using automated high-throughput manufacturing processes, according to an aspect of the present disclosure.

### DESCRIPTION OF THE INVENTION

As used herein, the singular form of “a”, “an”, and “the” include plural referents unless the context clearly states otherwise.

As used herein, the terms “right”, “left”, “top”, “bottom”, and derivatives thereof shall relate to the invention as it is oriented in the drawing figures. The term “proximal” can refer to a portion of an object or device that is manipulated by a user and/or which is located farthest away from a target. By contrast, the term “distal” can refer to a portion of the object or device that is farthest away from the user or closest to the target of the object or device. For example, a “proximal end” of a catheter can refer to an end of a catheter outside of the patient's body, which can include a hub configured to be manipulated by a user. By contrast, the “distal end” of a catheter can refer to the end of the catheter that is implanted in the patient's body, such as within a vessel or organ of the patient. However, it is to be understood that the invention can assume various alternative orientations and, accordingly, such terms are not to be considered as limiting. Also, it is to be understood that the invention can assume various alternative variations and stage sequences, except where expressly specified to the contrary. It is also to be understood that the specific devices and processes illustrated in the attached drawings, and described in the following specification, are examples. Further, depicted elements are not necessarily to scale, but are depicted in a manner to facilitate the showing of any described element and its relation to other elements of a described device. Hence, specific dimensions and other physical characteristics related to the embodiments disclosed herein are not to be considered as limiting.

For the purposes of this specification, unless otherwise indicated, all numbers expressing, for example, dimensions, physical characteristics, and so forth used in the specification and claims are to be understood as being modified in all instances by the term “about.” Unless indicated to the contrary, the numerical parameters set forth in the following specification and attached claims are approximations that can vary depending upon the desired properties sought to be obtained by the present invention.

Notwithstanding that the numerical ranges and parameters setting forth the broad scope of the invention are approximations, the numerical values set forth in the specific examples are reported as precisely as possible. Any measured numerical value, however, may inherently contain certain errors resulting from the standard deviation found in their respective testing measurements.

Also, it should be understood that any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1.

As used herein, the terms “comprising”, “comprise”, or “comprised”, and variations thereof, are meant to be open ended.

As used herein, the term “patient” refers to members of the animal kingdom including but not limited to human beings.

As used herein, the terms “communication” and “communicate” refer to the receipt or transfer of one or more signals, messages, commands, or other types of data. For one unit or component to be in communication with another unit or component means that the one unit or component is able to directly or indirectly receive data from and/or transmit data to the other unit or component. This can refer to a direct or indirect connection that can be wired and/or wireless in nature. Additionally, two units or components can be in communication with each other even though the data transmitted can be modified, processed, routed, and the like, between the first and second unit or component. For example, a first unit can be in communication with a second unit even though the first unit passively receives data and does not actively transmit data to the second unit. As another example, a first unit can be in communication with a second unit if an intermediary unit processes data from one unit and transmits processed data to the second unit. It will be appreciated that numerous other arrangements are possible.

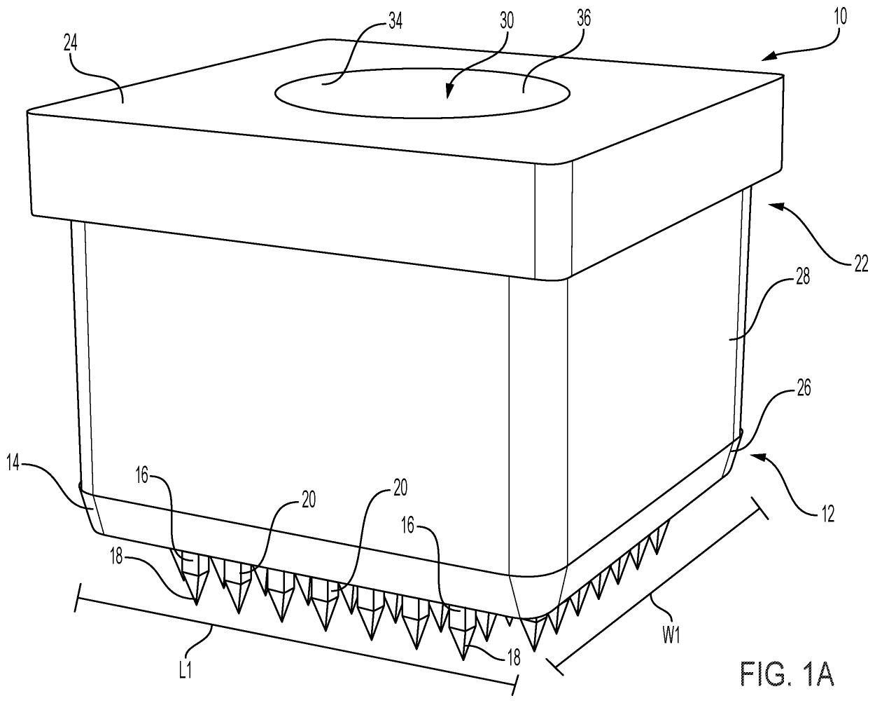

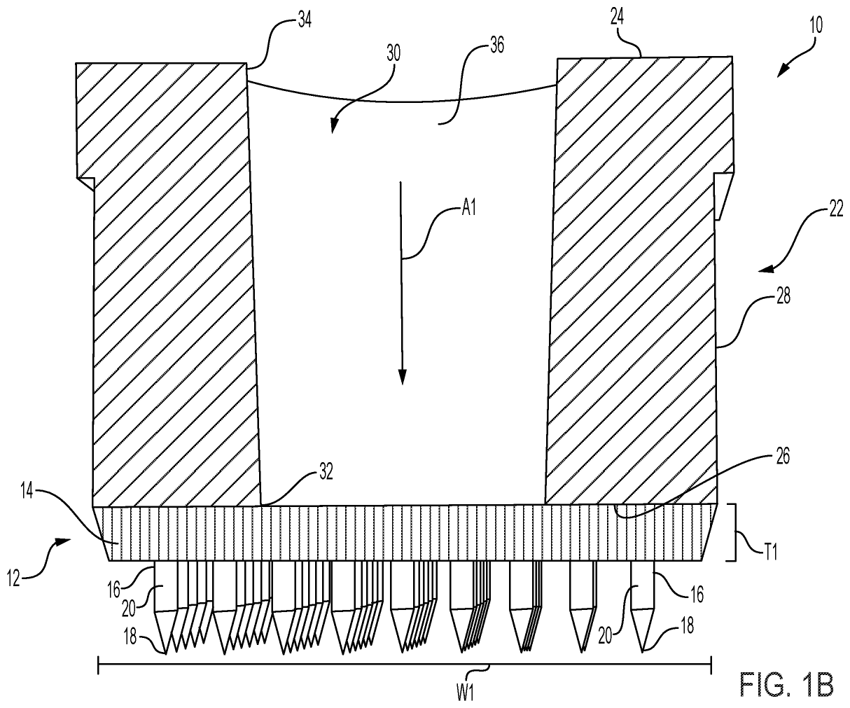

The present disclosure is directed to methods and systems 210 for manufacturing various types and configurations of microneedle arrays (MNAs), such as the exemplary MNA's shown in FIGS. 1A and 1B. The present inventors have recognized that MNAs 10 can be an excellent vaccination strategy for delivering vaccines into the skin (e.g., via an intradermal (ID), transdermal, or buccal route). While not intending to be bound by theory, it is believed that skin may have the most sophisticated immunologic network of any body tissue thanks to its rich population of powerful antigen-presenting cells (APCs) and other immune-accessory cells that rapidly elicit potent, long-lasting, and antigen-specific immune responses. In particular, studies have demonstrated that ID vaccinations trigger an immune response significantly more effectively than IM vaccinations, reducing the required dose by up to 100 times. However, despite its potential advantages, the present inventors believe that ID vaccination has not been utilized broadly due to the lack of reliable, reproducible, and simple administration techniques. Further, some current methods for ID injection require a highly trained clinician to administer the ID injection correctly. Even for properly trained clinicians, current ID injection methods require significant hand dexterity, skill, and experience. Due to these factors, as currently implemented, ID delivery is not a very reproducible method. Accordingly, new ID delivery strategies are needed to deliver vaccines effectively and efficiently to human skin.

The present inventors have previously demonstrated that dissolvable MNAs 10 can effectively deliver bioactive agent(s) to skin in vivo in animal and human studies. It is believed that dissolvable MNAs and other types of MNAs, such as through-hole, coated, or hybrid MNAs, can be used to facilitate reproducible delivery of a high concentration of antigen to skin microenvironments for efficient and effective presentation of antigens to the skin's immune cells. Recent studies have also shown that drugs and other therapeutic agents can be delivered through the skin using MNAs 10. For example, studies have shown that MNAs 10 can comprise or include drugs and other agents for treating skin diseases and conditions (e.g., squamous cell carcinoma, basal cell carcinoma, melanoma, and other skin cancers, inflammatory skin diseases such as psoriasis, cosmetic delivery such as Botox, or hair growth treatments). MNAs 10 can also be used for the delivery of therapeutic agents that provide systemic effects, such as pain-relief and anti-inflammatory drugs, stem cells, and other cell therapies. Currently known or future developed MNAs 10 may also be prime candidates for delivering gene therapy, stem-cell therapy, and cancer therapy applications.

The present inventors have recognized that in order to create clinically relevant and marketable MNA 10 medical devices for vaccination and other drug delivery uses, scalable, high-throughput, precise, reproducible, and flexible manufacturing techniques are needed that can fabricate MNAs 10 within a Good Manufacturing Practice (GMP) environment at sufficient scales (i.e., production volumes) to address the application demands. Current MNA fabrication techniques involve multiple manual steps. While such manual fabrication approaches may provide MNAs for research and laboratory use, these approaches are believed to be insufficient for the delivery of therapeutic agents for large populations. For example, for vaccination applications (e.g., coronavirus vaccination), a production rate of billions of MNAs per year may be needed. Current MNA fabrication techniques involving multiple manual steps considerably limit scalability and GMP alignment, meaning that current fabrication techniques will not produce the number of MNAs needed for mass vaccination and other large-scale efforts.

In order to address such application demands, the systems 210 and methods of the present disclosure provide for scalable, high-throughput, precise, reproducible, and flexible manufacturing of MNAs 10. In some examples, the manufacturing methods disclosed herein can be fully automated using a system 210 comprising several electromechanical movers, such as conveyors (e.g., conveyor belts or rollers), robots, and similar devices, for moving molded parts through a manufacturing unit and/or for assembling molded parts to produce the MNAs 10. The electromechanical movers and other automated components of the systems 210 of the present disclosure can be used along with automated manufacturing processes to allow for continuous production of MNAs 10. Using such automated processes and continuous production, it is believed that MNAs 10 may be produced at a rate of 75,000-100,000 or more MNAs per day by a single manufacturing unit or cell that takes up approximately 150 sq. ft. of space. As an example, a factory may have one hundred or more manufacturing units or cells, meaning that the factory may be able to produce 10 million MNAs per day.

In some examples, the systems 210 and methods of the present disclosure can be used for manufacturing different types of MNAs 10, such as MNAs 10 having fully or partially dissolvable or biodegradable microneedles, hollow (thru-hole) microneedles, and/or coated (solid needles with a drug coating) microneedles. Recently, MNAs 10 with hybrid (or hybrid-thru-hole) microneedles (“hMNAs”) have also been developed. As described in further detail herein, the systems 210 and methods of the present disclosure can be modified for manufacturing hMNAs, where only the sharp tips of the microneedles are dissolvable, biodegradable, or bioerodible, while other portions of the microneedles either dissolve considerably slower than the tips or do not dissolve or degrade at all.

By “biodegradable or “bioerodible,” it is meant that a polymer or another material, once implanted and placed in contact with bodily fluids and tissues, will degrade cither partially or completely through chemical reactions with the bodily fluids and/or tissues, typically and often preferably over a time period of minutes, hours, days, weeks, or months. Non-limiting examples of such chemical reactions include acid/base reactions, hydrolysis reactions, and enzymatic cleavage. The biodegradation rate of the polymer matrix may be manipulated, optimized, or otherwise adjusted, so that the matrix degrades over a useful time period. The polymer or polymers typically will be selected so that it degrades in situ over a time period to optimize mechanical conditioning of the tissue. Another class of polymers, which can be uses with the MNAs 10 disclosed herein are dissolvable polymers. As used herein, “dissolvable” polymers refer to polymers that dissolve into a liquid form and, while the polymers may not chemically degrade, such polymers can be biocompatible and can turn into a molecular form, which can be expelled from the body after use.

In some examples, the microneedles of the hMNAs 10 can comprise micro-cannula or hollow “tubes” attached to the sharp tips. As used herein, “tubes” or “micro-cannula” can refer to structures (e.g., pillars) with a bore at their center to facilitate transporting a liquid or gel. The micro-cannula or tubes can be formed from materials that are not dissolvable or biodegradable or from materials that dissolve significantly slower than the tips of the microneedles. hMNAs 10 can be configured such that fluid therapeutic agents can be delivered to skin through the micro-cannula after the tips dissolve or degrade. In some examples, hMNAs 10 further comprise an adapter for attaching a fluid delivery device, such as a syringe, or another fluid reservoir to the hMNA 10 for delivering a fluid therapeutic agent to a patient's skin through the micro-cannulas of the hMNA 10.

### Hybrid Microneedle Arrays (hMNAs)

As previously described, the manufacturing systems 210 and methods of the present disclosure can be used for manufacturing MNAs 10 with different types of microneedles, including dissolvable, hollow (thru-hole), and/or coated (solid with drug coating) microneedles. The systems 210 and methods of the present disclosure can also be used for making hMNAs, such as exemplary hMNAs 10 shown in FIGS. 1A and 1B. Other exemplary hMNAs that can be manufactured by the systems 210 and methods of the present disclosure are described in PCT Appl. Pub. No. WO 2021/207705A1, entitled “Hybrid microneedle arrays,” which is incorporated herein by reference in its entirety.

As shown in FIGS. 1A and 1B, an hMNA 10 comprises a molded part 12 formed from a biocompatible material comprising a base 14 and multiple microneedles 16 extending from a distal surface of the base 14. For example, a 7 mm×7 mm MNA 10 may have approximately 25-150 microneedles extending from the base 14. As described herein, the molded part 12 is formed from a biocompatible material that does not dissolve or degrade or which dissolves or degrades slowly compared to the hydrogel material that forms the tips of the microneedles 16. For example, the base 14 can be a substantially flat substrate or plate formed from a cured (e.g., UV curable) polymer resin having a length L1 and/or width W1 ranging from about 2 mm to about 50 mm, and a thickness T1 ranging from about 0.2 mm to about 2 mm. The base 14 can be square shaped, rectangular, circular, elliptical, or any other convenient shape. As used herein, a “microneedle” can refer to a needle having a length ranging from about 50 μm to about 5000 μm and a width or diameter ranging from about 50 μm to about 500 μm. The MNAs 10 disclosed herein can comprise a single microneedle or as many as 100 s of microneedles.

The microneedles 16 can have a circular cross-section, a square-shaped cross-section, or any other convenient cross-sectional shape depending, for example, on the desired deployment location and/or type of flowable material being delivered by the hMNA 10. As described in further detail herein, the microneedles 16 can be solid or hollow comprising micro-cannula 20 extending at least partially through the microneedles 16. The microneedles 16 can have a sharped distal tip 18 configured to pierce skin (or other tissue) and a proximal end connected to and/or integrally formed with a distal surface of the base 14. The distal tip 18 can have a variety of shapes depending, for example, on desired skin penetration depth and/or on materials used for forming the distal tip 18 and/or micro-cannula 20. In some examples, the distal tip 18 can be a pyramid, cone, arrowhead, triangular, incurvate, or ovate-shaped tip. Further, the size of the distal tip 18 may vary and, in some examples, a diameter or width of the distal tip 18 can be larger than the micro-cannula 20 to create an undercut or a temporary retaining feature. The microneedles 16 can be arranged in a pattern or array of rows and columns, a concentric circular pattern, or can be more randomly distributed on the distal surface of the base 14.

For dissolvable or hollow (thru-hole) MNAs 10, the entire molded part 12 including both the base 14 and the microneedles 16 can be formed from the same material. For example, the entire molded part 12 can be formed from dissolvable or biodegradable polymers, such as carboxymethyl cellulose (CMC), and polyvinyl alcohol (PVA) hydrogel, or combinations thereof. In other examples, materials for forming the dissolvable needles or distal tips 18 can comprise simple sugars, such as glucose, trehalose, maltose, or dextrose, as well as polymers such as natural or synthetic polysaccharides, such as hyaluronic acid, maltodextrin, poly(lactic acid) (PLA), Poly(D,L-lactide-co-glycolide) (“PLGA”), poly(glycolic acid) (PGA), polyethylene glycol (PEG), polyethylene oxide (PEO), or Polyvinylpyrrolidone (PVP), among many others. Different combinations of these polymers may be used to provide desirable mechanical strength and/or dissolution time, as well as in consideration of the drug to be delivered (e.g., to provide long-term stability or shelf life to the drug).

For a coated MNA 10, the base 14 and the microneedles 16 can be formed from a non-dissolvable or slow-dissolving polymer material, which is coated by a different material, such as a therapeutic agent mixed with a dissolvable and/or biodegradable material. By contrast, each microneedle 16 on an hMNA 10 can comprise a distal tip 18 formed from a dissolvable and/or biodegradable material, such as the previously described polymers, and a stem or micro-cannula 20 comprising a non-dissolvable material, such as a UV curable, biocompatible resin, or a dissolvable material that dissolves far slower than the distal tip 18. The micro-cannula 20 can comprise a thru-hole at the center of its cross-section, which extends through the microneedle 16 and base 14 of the hMNA 10.

The hMNA 10 can further comprise an auxiliary component, such as an adapter 22 or a separate backing. The adapter 22 can be formed from a non-dissolvable material, such as the UV curable polymer resin. As shown in FIGS. 1A and 1B, the adapter 22 can be a box or cube-shaped structure having a proximal surface 24, a distal surface 26, a sidewall 28, and a through-hole 30 having a circular or substantially circular cross-sectional shape, extending through the adapter 22. The through-hole 30 can comprise an open distal end 32, an open proximal end 34, and an inner surface 36 extending therebetween. The through-hole 30 can be straight, tapered, or threaded to accommodate standard syringe tips of a fluid delivery device. Fluid from the fluid delivery device passes through the through-hole 30 of the adapter 22, as shown by the arrow A1 (in FIG. 1B), and then through the micro-cannula 20 of the microneedles 16 for delivery to the patient. In other examples, the through-hole 30 can also be configured to connect to a self-contained “pouch” ejector, which can be actuated to deliver medical fluid from the pouch through the through-hole 30.

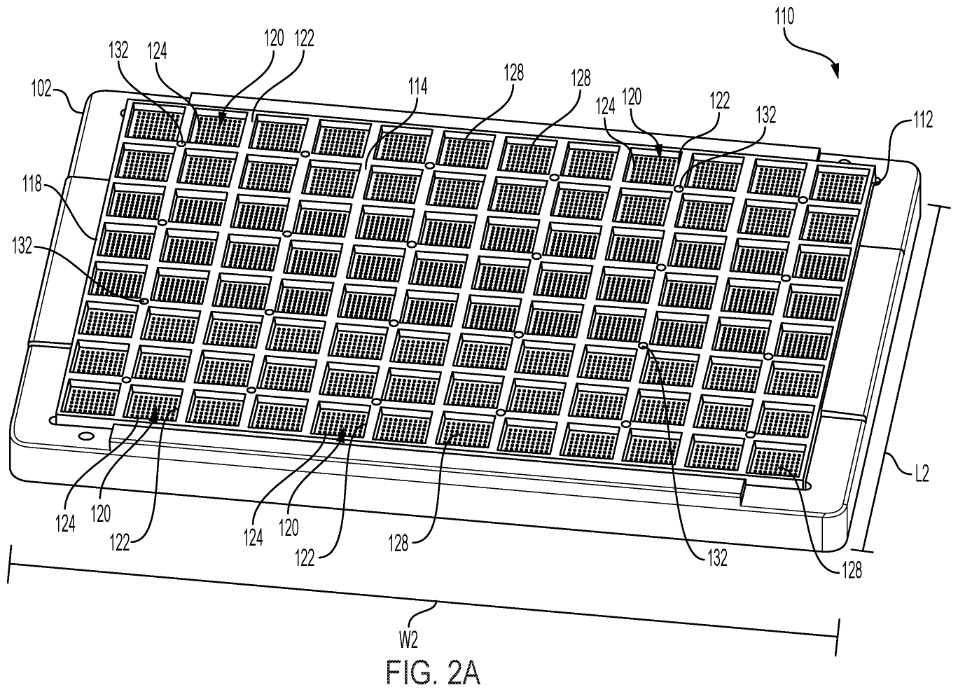

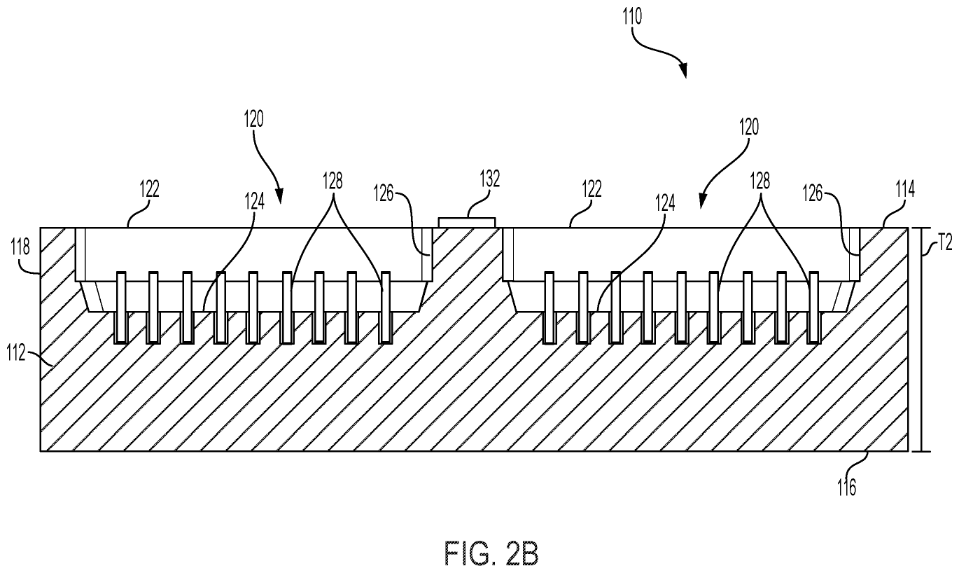

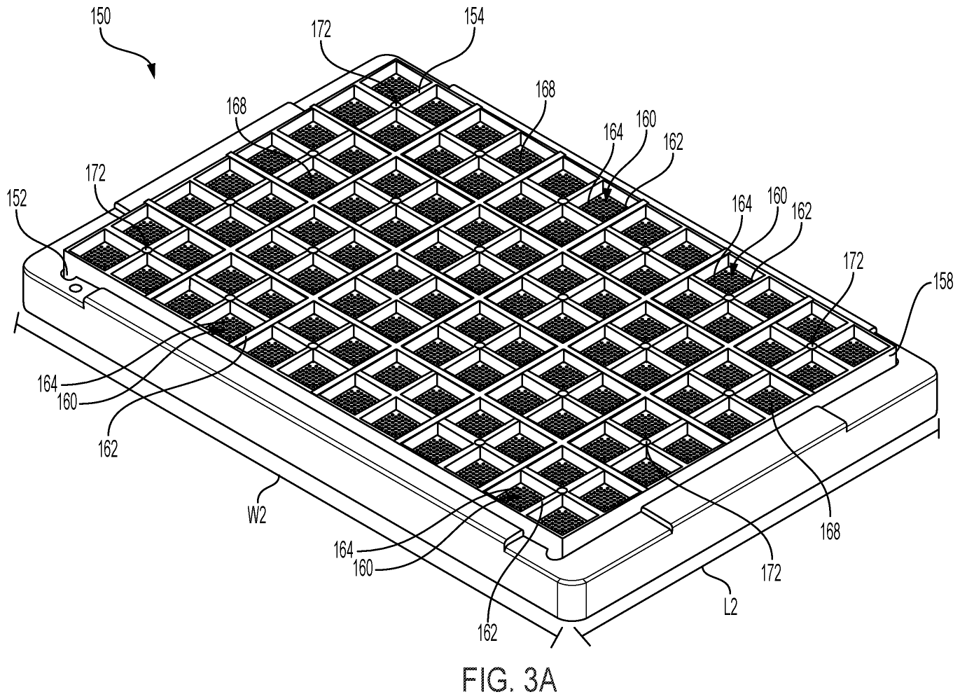

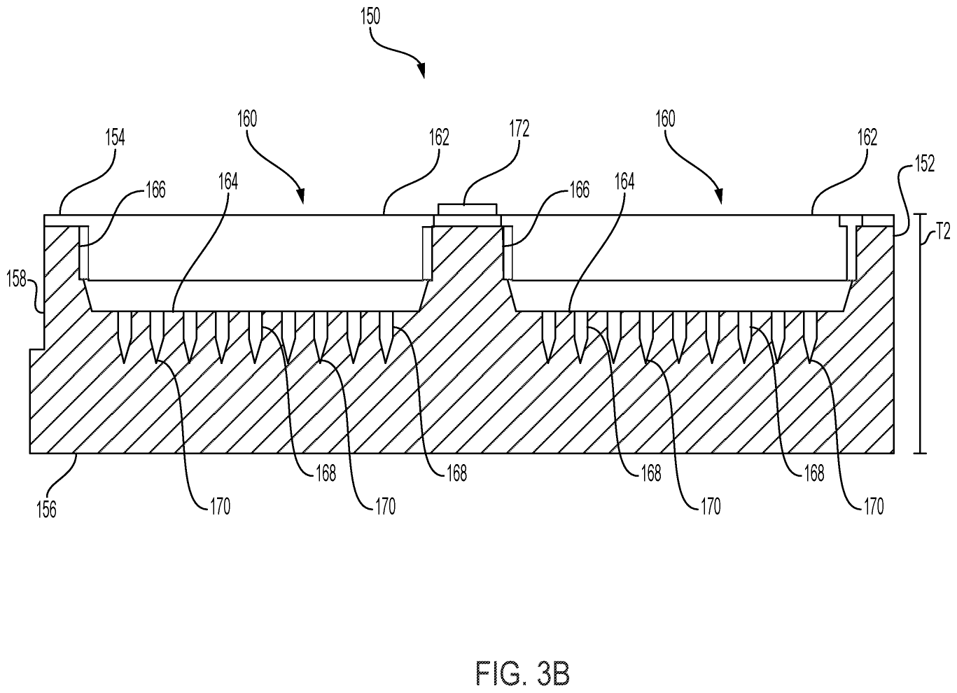

The adapter 22 or other auxiliary components can be made independently, provided that dimensional accuracy and material characteristics required by integration/precision assembly with the hMNAs 10 are satisfied. For example, depending on the geometry, the adapter 22 can be fabricated separately using injection molding, spin casting, 3D printing, or other resin/plastic manufacturing processes, as are known in the art. Alternatively, the adapter 22 can be molded from, for example, polymer resin along with other components of the hMNAs 10. As described in further detail herein, at some point during the manufacturing process, the adapter 22 is assembled with the base 14 of the hMNA 10. In some examples, assembly occurs as an integral part of the manufacturing process. For example, the adapter 22 can be placed into a mold (such as the micro-cannula mold 110, shown in FIGS. 2A and 2B or the tip mold 150 shown in FIGS. 3A and 3B) before dispensing a biocompatible polymer resin or dissolvable hydrogel into the molds 110, 150. When the polymer resin or hydrogel cures or dries, it also adheres to the adapter 22, creating a single unit that can be used as an independent device. In other examples, a thicker, dissolving, or non-dissolving coating or backing can be assembled on the proximal surface of the MNA 10 instead of the adapter 22. The non-dissolving coating can be removed from the patient's skin after dissolvable portions of the MNA 10 (e.g., the microneedles 16 and/or base 14) dissolve or degrade. By contrast, dissolvable, hollow, and coated MNAs do not require an adapter 22; however, a separate backing layer to facilitate administration can be fabricated by the manufacturing systems disclosed herein or can be made separately and assembled onto the MNAs 10 after molding.

In other examples, the hMNA 10 can comprise an applicator or a stand-alone reservoir/blister pouch instead of the adapter 22. For example, a reservoir/blister pouch can be coupled to the base 14 of the hMNA 10. The reservoir/blister pouch can be configured to be manually deformed for rapid delivery of a fluid therapeutic agent through tubular segments or the micro-cannula 20 of the hMNA 10. In other examples, the reservoir or pouch can be configured to deform more slowly for delivery of the therapeutic agent over a longer period of time.

### High-Capacity Molds for MNAs

High-capacity production molds 110, 150 for rapidly producing large numbers of MNAs 10 for mass vaccination and other medical uses are shown in FIGS. 2A-3B. Specifically, high-capacity micro-cannula molds 110 are shown in FIGS. 2A and 2B for making the molded parts 12 comprising the micro-cannula 20. High-capacity tip molds 150 for making biodegradable or dissolvable tips of the hMNAs 10 are shown in FIGS. 3A and 3B. The micro-cannula molds 110 can also be used for making hollow (thru-hole) MNAs. The tip molds 150 can also be used for making dissolvable MNAs and/or coated MNAs, including solid microneedles 16 coated with a drug or another therapeutic agent. In some examples, the production molds 110, 150 can be mounted to plates 102 for supporting the molds 110, 150 and/or for engaging movers and other components of the systems 210 for moving the molds 110, 150 through the system 210.

As described in further detail herein, the molds 110, 150 can be made from micro-machined master molds made from hard plastic, metal, or other durable materials. For example, the master mold can be a standardized acrylic plate with precision holes and edges for handling and assembly. The production molds 110, 150 used for making the MNAs 10 can comprise an elastomer, such as siloxanes (e.g., polydimethylsiloxane (PDMS)), a thermoplastic, or a light-curable resin. Other materials, such as resin, may be also used for making production molds. The production molds 110, 150 can be made in the master molds and then removed from the master molds for use in making the MNAs 10. In some examples, the production molds 110, 150 can be reused many times for making multiple batches of MNAs 10.

FIGS. 2A and 2B show the micro-cannula production mold 110 including features for creating a through-hole system. Specifically, as shown in FIGS. 2A and 2B, the micro-cannula mold 110 comprises a flexible plate or tray 112 comprising a top surface 114, a bottom surface 116, and a sidewall 118 extending between the top surface 114 and the bottom surface 116. As described in further detail herein, dimensions of the mold 110 can be selected so that the mold 110 can be used with existing automated manufacturing machines, assembly robots, and similar devices. For example, tray 112 can have the following dimensions: a width W2 of about 20 mm to about 300 mm or about 100 mm to 150 mm; a length L2 of about 20 mm to about 300 mm or about 50 mm to 100 mm; and a thickness T2 of about 2 mm to about 30 mm or about 5 mm to 15 mm. In order to produce molded parts 12 with sufficiently high precision to assemble the hMNAs 10, the molds 110 desirably are made with an overall precision better than 20 μm, and resolution better than 5 μm.

The micro-cannula mold 110 is divided into wells 120 on the top surface 114 of the tray 112. Dimensions of the wells 120 can be determined based on a desired final size of the MNA or hMNA patches 10 and/or the number of microneedles 16 of the MNA 10. For example, the wells 120 can be sized to make MNAs 10 with a length and/or width of about 2 mm to about 25 mm and having from 1 microneedle to about 1000 microneedles. Each well 120 can comprise an open top 122, a closed bottom 124, and an inner surface 126 extending between the open top 122 and the closed bottom 124 of the well 120. Each well 120 can be sized to form a single molded part 12, which can be assembled into the MNA 10. In some examples, the mold 110 comprises about 8 wells to about 1000 wells. In one specific example, the large-capacity mold 110 includes 96 wells 120 and is the same size as a standard 96-well plate used for biological sampling. This “standard” 96-well sizing means that existing biomedical sampling equipment, such as dispensing and centrifuge systems, can be used with the systems 210 of the present disclosure. Accordingly, the systems 210 can be easily assembled from existing parts and devices and may not require fabrication of customized parts and machines.

As shown in FIGS. 2A and 2B, the micro-cannula molds 110 further comprise multiple holes in the closed bottom 124 of the wells 120 with posts 128 extending from a distal end of the holes towards the top 122 of the wells 120 for forming the micro-cannula, lumens, or fluid channels of the microneedles 16. The posts 128 can have a circular cross-section, a square-shaped cross-section, or other convenient shapes. The posts can be about 50 μm to about 3500 μm in total length and can extend proximally from the bottom 124 of the well 120 by a distance of from about 100 μm to about 2500 μm. Each well 120 can include from about 25 posts to about 150 posts 128. In some examples, the posts 128 are arranged in rows and columns, with a post 128 being aligned with an adjacent post 128 in both a width and length direction. The posts 128 can also be arranged in a concentric circular pattern or can be randomly distributed in the wells 120.

FIGS. 3A and 3B show the tip mold 150, which can be used for making a molded part 12 with dissolvable needles and/or for making biodegradable or dissolvable tips 18 to be attached to micro-cannula 20 of the hMNA 10. As with the micro-cannula mold 110 of FIGS. 2A and 2B, the tip mold 150 comprises a flexible plate or tray 152 comprising a top surface 154, a bottom surface 156, and a sidewall 158 extending between the top surface 154 and the bottom surface 156. The tray 152 is mounted to the plate 102, which can be configured to be engaged by conveyors (e.g., conveyor belts or rollers) or other electromechanical movers of the system 210. The tray 152 can be similar or identical in size and shape to the tray 112 of the micro-cannula mold 110. The tray 152 can be divided into wells 160 on the top surface 154 of the tray 152. Each well 160 can comprise an open top 162, a closed bottom 164, and an inner surface 166 extending between the open top 162 and the closed bottom 164 of the wells 160. When used to make hMNAs 10, the wells 160 of the tip mold 150 are generally identical in size and shape to wells 120 of the micro-cannula molds 110 so that molded parts 12 from the micro-cannula mold 110 and from the tip mold 150 can be assembled together to form the hMNAs 10.

The tip mold 150 further comprises one or multiple holes 168 on the closed bottom 164 of the wells 160 for forming microneedles 16 and/or dissolvable tips 18 of the hMNAs 10. The holes 168 can have a circular, elliptical, square, or rectangular cross-sectional shape. The holes 168 are generally arranged in patterns matching the posts 128 of the micro-cannula mold 110. For example, each well 160 can comprise from about 25 holes to about 150 holes 168. The holes 168 of each well 160 can be arranged in rows and columns, with each hole 168 being aligned with an adjacent hole 168 in both a width and length direction, or in a concentric circular pattern. Further, bottom or distal portions 170 of the holes 168 can be tapered for forming sharpened needle tips 18 in the holes 168.

In some examples, the wells 120, 160 of the micro-cannula mold 110 and/or the tip mold 150 can comprise or be coated with a super-hydrophobic coating over the closed bottoms 124, 164 and/or inner surfaces 126, 166 of the wells 120, 160 to facilitate demolding of molded parts 12 from the molds 110, 150. For example, the super-hydrophobic coating can comprise a biocompatible polymer with low surface energy, such as fluorinated chlorosilanes. An exemplary fluorinated chlorosilane that can be used in the wells 120, 160 of the present disclosure is Trichloro(1H,1H,2H,2H-perfluorooctyl) silane (“PFOCTS”). In other examples, the coating can comprise a Parylene coating, which can be deposited by sputtering.

In some examples, the micro-cannula mold 110 and/or the tip mold 150 further comprise fiducial or contact markers, precision holes, or similar identifiers to allow for camera or contact-based alignments. For example, the molds 110, 150 can comprise fiducial markers 132, 172 on the trays 112, 152. The fiducial markers 132, 172 can be easily identifiable shapes (e.g., circles, triangles, squares) molded onto and/or protruding from the top surfaces 114, 154 of the trays 112, 152. In some examples, fiducial markers 132, 172 can be painted or coated to be a different color from other portions of the trays 112, 152 to make the fiducial markers 132, 172 easier to identify in captured images of the trays 112, 152. The fiducial markers 132, 172 can be provided so that the molds 110, 150 can be aligned and/or correctly positioned relative to components and devices of the MNA manufacturing systems 210 for automated MNA manufacturing. For example, the fiducial markers 132, 172 can be configured to be identified in images of the molds 110, 150 captured by an optical sensor, camera, or camera system of the MNA manufacturing system 210 for aligning the mold 110, 150 with a dispenser (e.g., a dispenser for dispensing polymer resin into the wells 120, 160) or other components (e.g., assembly robots, curing devices, etc.) of the MNA manufacturing system 210.

While injection molding is often used for making a variety of single-use or disposable objects for rapid manufacturing processes, injection molding may pose substantial challenges in making the molds 110, 150 of the present disclosure. Therefore, the following two-step spin-casting process can be used for making the molds 110, 150. In a first step, as previously described, a high-precision large-sized master mold is created from hard plastic, ceramic, silicon, metal, or other rigid and durable materials. The master molds can be created using various machining and/or fabrication processes, as are known in the art, such as precision micro-milling/milling, lithography/clean-room microfabrication/photolithography, micro-electro-discharge machining, micro-electrochemical machining, or other precision manufacturing processes. Desirably, the selected machining and/or fabrication process should be capable of providing strict geometric accuracy (within ˜1 μm) and, when applicable, should provide sufficient tip sharpness (e.g., less than 5 μm tip radius). In one approach, the master mold can be fabricated using mechanical micro-machining processes, including micro-milling and micro-drilling on a high-precision system. The master mold materials, such as hard plastics (e.g., acrylic) or metals (e.g., brass), can be used to strike a balance of machinability, obtaining high-quality features and smooth surfaces (for molding and demolding preparations), and durability. Tungsten carbide or diamond tools can be used to create the master molds. Each master mold can be used hundreds or thousands of times without wear or deterioration.

In a second step, the master molds are then used to create the production molds (e.g., the previously described micro-cannula mold 110 and the tip mold 150) from an elastomer or a resin using polymer molding techniques, such as injection molding or polymer (e.g., spin) casting. For example, the production molds 110, 150 can be made from an clastic material with good feature-replication capability, such as siloxanes, e.g., polydimethylsiloxane (PDMS). The molds 110, 150 can be alternatively made from thermosets, thermoplastics, or light-curable resins. This molding process may require the use of a centrifuge and/or vacuum to ensure that the micro-scale features of the molds 110, 150 are well replicated. Each production mold 110, 150 can be reused many times (e.g., hundreds of times). During this molding step, additional components (such as side frames, etc.) can be connected or adhered to the molded part 12. Demolding large PDMS or other molds from the master mold can be challenging. To facilitate this demolding process, the master mold and/or the production molds 110, 150 can be coated with a biocompatible polymer with low surface energy (e.g. fluorinated chlorosilanes, such as PFOCTS), creating super-hydrophobic surfaces of the master mold and/or the production molds 110, 150. In some examples, a demolding system can be used, wherein a titanium grid disposed within the master mold is used, along with a hydraulic press, to demold the production molds 110, 150 from the master molds uniformly and without damaging the production molds 110, 150. Alternatively, demolding can be performed by placing the master mold with the cured production mold 110, 150 (e.g., the PDMS production molds) connected thereto, upside down into a centrifuge bucket. The master mold can be placed on top of spacers so that the molded parts are elevated from a bottom surface of the bucket, meaning that there is room for the production mold 110, 150 to drop down away from the master mold. When in operation, the centripetal applies uniform downward force to help dislodge the production mold 110, 150 from the master mold without damaging any of the molds 110, 150. During this demolding process, the centrifuge can be operated at a low speed, such as about 1000 rpm.

In other examples, production molds 110, 150 for use with the systems 210 of the present disclosure can be directly created using various micro-manufacturing processes, as are known in the art. For example, fabrication of the previously described production molds 110, 150 can be accomplished directly using molding, 3D printing, metal additive manufacturing, photolithography, or other processes. In general, due to the size and form of the features used in MNAs 10, this direct fabrication process for production molds 110, 150 can be challenging. For instance, current 3D printing processes may not provide the required combination of high accuracy, sharp tips, and smooth surfaces needed for complex mold/MNA structures, which can be achieved by the previously described two-step production method. The present inventors believe that a direct approach to making complex production molds 110, 150 may be possible using a combination of different currently-available fabrication techniques or using fabrication techniques with improved precision developed or refined in the future.

### High-Throughput Manufacturing Systems

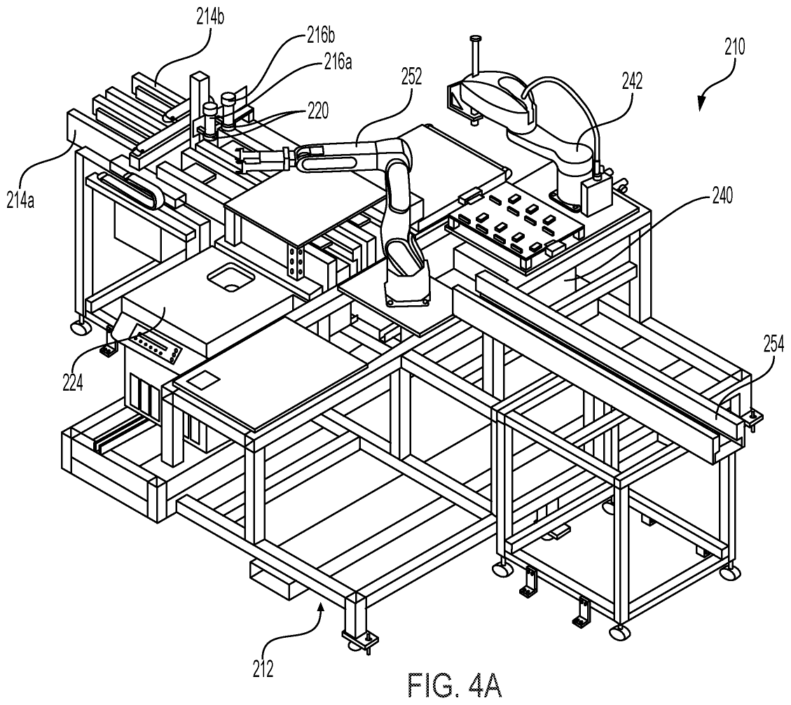

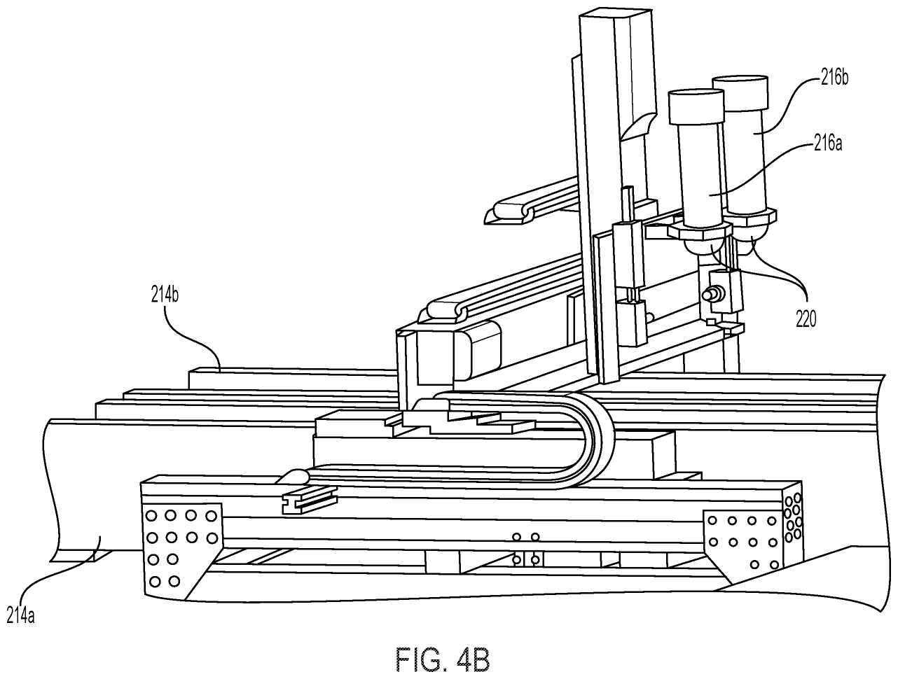

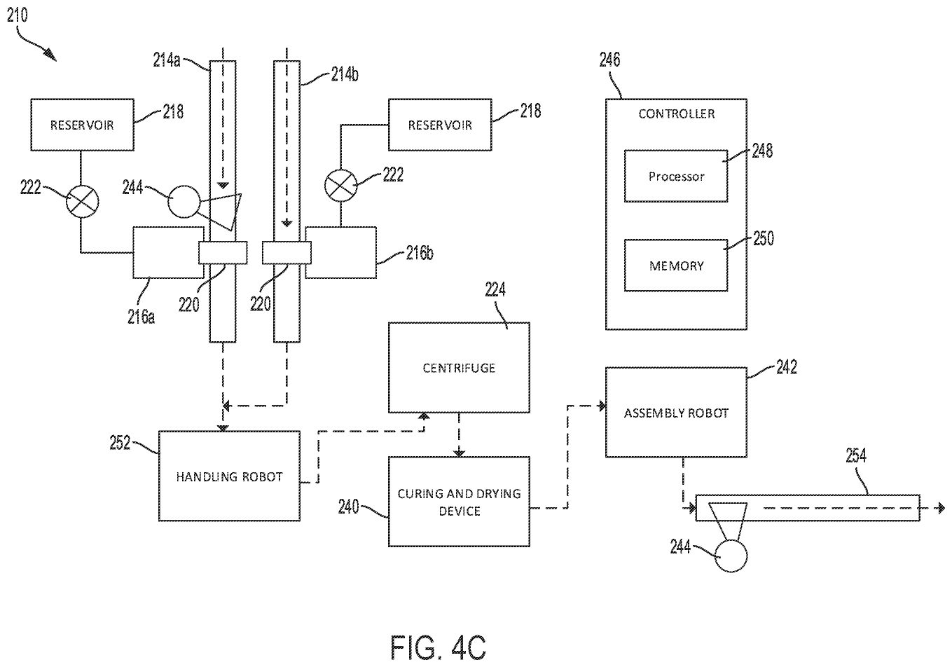

A high-throughput manufacturing system 210 for assembling MNAs 10 using one or more of the previously described molds 110, 150 is shown in FIGS. 4A and 4B. The system 210 can be a single manufacturing unit or “unit cell” comprising all fabricating components, handling and assembly components, and computer and/or processing circuitry required for automated manufacturing of MNAs 10 using the molds 110, 150. As previously described, a factory can include many manufacturing units or unit cells for manufacturing millions or tens of millions of MNAs 10 per day.

As shown in FIG. 4A, components of a single manufacturing cell or unit can be mounted to a single support frame 212, such as a frame comprising multiple vertical and horizontal members for supporting components of the system 210. The frame 212 can comprise multiple locations for installing different system components, which allows for flexibility in how system components are arranged and used. For example, the frame 212 can include locations for installing multiple centrifuges. In some examples, the manufacturing unit or cell can be set up with only one centrifuge for spin casting the MNAs 10 in the molds 110, 150. As described in further detail herein, in other implementations, the manufacturing unit or cell can be set up with a first centrifuge connected to a first location of the support frame 212 for spin casting the molds 110, 150 and a second centrifuge connected to a second location of the frame 212 that can be used for drying polymer resin or hydrogel of assembled MNAs 10.

With continued reference to FIGS. 4A-4C, the system 210 further comprises electromechanical movers for moving the molds 110, 150 through the system 210. As used herein, a “mover” refers to an electromechanical device controlled by a computer processor that moves molds 110, 150 between components of the system 210. A “mover” can also refer to system components that remove molded parts 12 from the molds 110, 150 after curing and/or drying, as well as to system components that assemble molded parts 12 to form assembled MNAs 10. Electromechanical movers can include various devices commonly used in warehouses, factories, and other assembly line systems, such as motors, linear actuators, mechanical lifts, conveyors (e.g., conveyor belts, rollers, vibratory conveyors), assembly robots, handling robots, robotic arms (e.g., a four-axis or six-axis arm), pick and place machines, and similar devices, as are known in the art.

In some examples, the electromechanical mover can comprise one or more input high-precision conveyors 214a, 214b for moving empty mold(s) 110, 150 towards other devices of the system 210. The conveyors 214a, 214b can comprise belts, rollers, or other units for moving small objects, as are known in the art. To facilitate connection with the conveyor 214a, 214b, the mold(s) 110, 150 can be mounted to the standardized base plates 102 (shown in FIGS. 2A and 3A), such as plates 102 made from a rigid plastic material (e.g., acrylic). The base plate 102 can be configured to be engaged by the conveyors 214a, 214b and other movers of the system 210 to facilitate accurate placement and handling of the molds 110, 150 throughout the fabrication and assembly processes.

A system for making hMNAs 10, as shown in FIGS. 4A-4C, can include two input conveyors 214a, 214b, such as a first conveyor 214a for moving the micro-cannula mold 110 towards components or stations of the system 210 and a second conveyor 214b for moving the tip mold 150 towards the components or stations of the system 210. The system 210 further comprises one or more dispensers 216a, 216b positioned at a dispensing station of the system 210. The input conveyor 214a, 214b can be configured to move the empty molds 110, 150 towards the dispensing station. The dispensers 216a, 216b can be configured to dispense a flowable polymer material into the wells 120, 160 of the empty molds 110, 150 for forming the molded parts 12. For example, the system 210 can comprise a first dispenser 216a for dispensing polymer resin (e.g., a UV curable polymer resin) to wells 120 of the micro-cannula mold 110. The system 210 can further comprise a second dispenser 216b for dispensing the dissolvable or biodegradable hydrogel or another polymer (i.e., a gel form of the polymer dissolved in a solvent such as water) into the tip mold 150 for making the dissolvable tips 18 of the hMNAs 10 or for making dissolvable microneedles 16 of other types of MNAs 10. As previously described, the dissolvable or biodegradable polymers can comprise carboxymethyl cellulose (CMC), polyvinyl alcohol (PVA) hydrogel, or combinations thereof. In other examples, materials for forming the dissolvable needles or needle tips 18 can comprise simple sugars, such as glucose, trehalose, maltose, or dextrose, as well as polymers such as natural or synthetic polysaccharides or polymers, including hyaluronic acid, maltodextrin, poly(lactic acid) (PLA), Poly(D,L-lactide-co-glycolide) (“PLGA”), poly(glycolic acid) (PGA), polyethylene glycol (PEG), polyethylene oxide (PEO), or Polyvinylpyrrolidone (PVP), among many others.

In some examples, the dispensing station is separate from the conveyors 214a, 214b. In that case, robotic arms or other mechanical mover and positioner devices, such as a handling robot 252, can be used for moving the empty molds 110, 150 from the conveyors 214a, 214b to the dispensing station and dispensers 216a, 216b. As shown in FIG. 4A, the handling robot 252 can be a four-axis or six-axis robotic arm mounted to the frame 212 for moving the molds 110, 150 between components of the system 210.

In other examples, the dispensers 216a, 216b can be placed on top of the conveyors 214a, 214b, as shown most clearly in FIG. 4B, so that no handling is required to move the empty molds 110, 150 from the input conveyors 214a, 214b to the dispensing station or dispensers 216a, 216b. Instead, the input conveyors 214a, 214b can be configured to stop when the molds 110, 150 are properly aligned under the dispensers 216a, 216b. Once the molds 110, 150 are in place and the input conveyors 214a, 214b are stopped, the dispensers 216a, 216b can be configured to dispense the polymer resin, dissolvable or biodegradable polymer, or hydrogel to appropriate or selected wells 120, 160. In some examples, the dissolvable or biodegradable hydrogel or another polymer can be loaded into wells 160 of the tip mold 150 as a high-concentration gel having a thick physical form. Due to the thickness of the gel, the molds 110, 150 may need to be agitated or otherwise manipulated to distribute the gel over surfaces of the wells 160, and so that the microneedle holes 168 (shown in FIGS. 3A and 3B) can be fully filled with the gel. Fully filling the holes 168 can also require loading a larger than required amount of dissolvable or biodegradable hydrogel or another polymer onto the wells 160. The excess polymer or hydrogel can be removed prior to curing. For example, a push-plate can be used to distribute the polymer or hydrogel over inner surfaces 166 of the wells 160 and/or for ensuring the polymer or hydrogel collects in the holes 168 for forming the distal tips 18 of the microneedles 16.

In some examples, the dispensers 216a, 216b can comprise reservoirs 218 containing the hydrogel, dissolvable polymer, and/or polymer resin. The reservoirs 218 can be fluidly connected to nozzles 220 of the dispensers 216a, 216b through conduits or connectors. Dispensing valves 222 can be positioned in the conduits or connectors for controlling fluid flow between the reservoirs 218 and the nozzles 220 for loading precise amounts of gel, dissolvable polymer, and/or resin into each well 120, 160 of the molds 110, 150. In some examples, the loading or fluid dispensing steps take about one second per well 120, 160. meaning that it takes about 96 seconds to dispense the polymer resin or hydrogel into each well 120, 160 of the molds 110, 150.

In some examples, the nozzles 220 can be a multi-head nozzle 220 having two, three, four, or more heads for dispersing multiple fluid streams to the wells 120, 160. In a preferred example, the nozzle 220 comprises four heads for ejecting polymer resin to four quadrants of the square or rectangular-shaped wells 120, 160. Multi-head nozzles 220 can also be used for filling multiple wells 120, 160 simultaneously.

In some examples, the system 210 comprises one dispenser 216a and nozzle 220 for dispensing polymer resins, drug-mixed gels, or other dissolvable or biodegradable polymer or hydrogel materials into the mold 110, 150. In particular, systems 210 for making dissolvable or hollow (thru-hole) MNAs 10 may only require a single input conveyor 214a and single dispenser 216a. Systems 210 for making hMNAs 10 (as shown in FIGS. 4A-4C) can include the two input conveyors 214a, 214b, along with the first dispenser 216a for dispensing polymer resin to the micro-cannula mold 110 and the second dispenser 216b for dispensing the dissolvable or biodegradable hydrogel or another polymer to the tip mold 150.

The system 210 further comprises a centrifuge 224 for centrifuging the molds 110, 150 causing the polymer resin material dispensed into the wells 120, 160 of the molds 110, 150 to distribute through the wells 120, 160. In some examples, the handling robot 252 can be configured to automatically load the filled molds 110, 150 into the centrifuge 224. The centrifuge 224 can be a custom-made centrifuge or a commercially available machine installed to the frame 212 of the system 210. The centrifuge 224 can be an automated or robotic centrifuge 224 that turns on and off or opens the lids and indexes without manual intervention. Further, the centrifuge 224 can be configured to automatically adjust operating parameters of the centrifuge 224 in order to ensure that the polymer resin, dissolvable polymer, or hydrogel are well distributed through the molds 110, 150 following centrifugation.

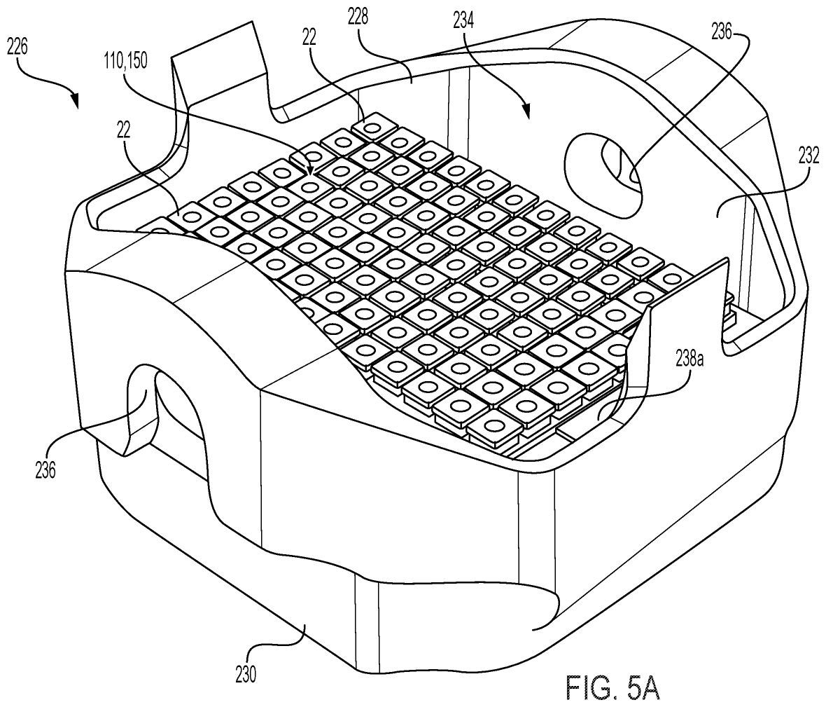

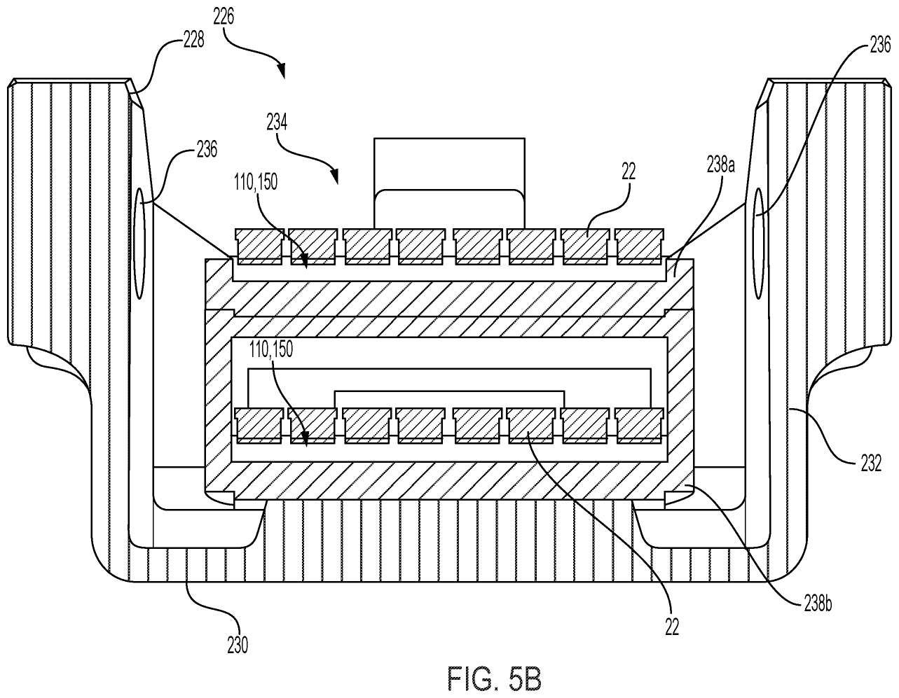

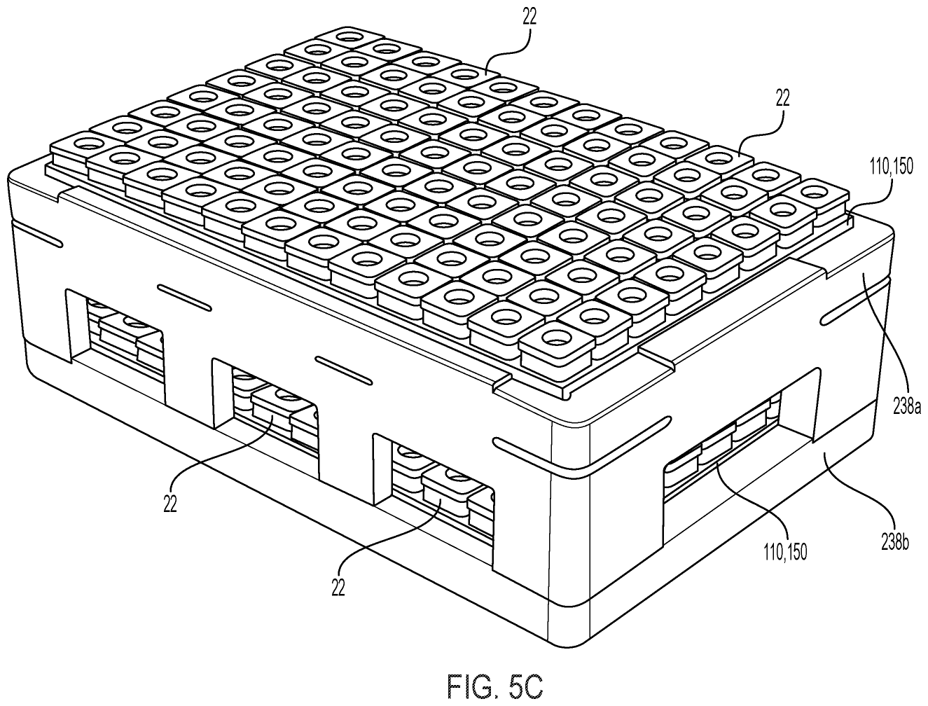

In some examples, the centrifuge 224 comprises or is configured to receive a support or “bucket” 226 containing one or more molds 110, 150 filled with the dispensed polymer material. Exemplary supports or buckets for the centrifuges 224 are shown in FIGS. 5A-5C. As shown in FIGS. 5A and 5B, the bucket 226 comprises an open top 228, a closed bottom 230, and a sidewall 232 extending between the open top 228 and the closed bottom 230. The sidewall 232 encloses a central cavity 234 or chamber sized to receive the molds 110, 150. The sidewall 232 can include openings 236 or through-holes configured for attaching the bucket 226 in a chamber of the centrifuge 224. The bucket 226 also includes platforms 238a, 238b in the central cavity 234 or chamber configured to engage the molds 110, 150 for holding the molds 110, 150 in place. For example, as shown in FIGS. 5B and 5C, the platforms 238a, 238b are configured to support two molds 110, 150. Specifically, the platform 238a, 238b comprises an upper shelf or platform 238a for receiving a first mold 110, 150, and a lower shelf or platform 238b for receiving a second mold 110, 150. As shown in FIGS. 5A-5C, the molds 110, 150 can be loaded with an adapter 22 positioned over wells 120, 160 of each mold 110, 150. As described in further detail herein, by centrifuging and distributing the polymer resin or other polymer material in the wells 120, 160 of the molds 110, 150, the adapters 22 can be adhered to the molded parts 12, thereby forming an MNA 10 with an adapter 22 adhered thereto.

With reference again to FIGS. 4A-4C, the system 210 further comprises a curing and/or drying device 240. The curing and/or drying device 240 can be a light or UV curing device, such as a UV cabinet or UV emitting bulb, for curing polymer resin in the micro-cannula mold 110. For example, the UV curing device can be a UV cabinet, such as a 100 W unit that uses UV-A light with a wavelength of 380 nm. The curing and/or drying device 240 can also be a thermal device, such as a furnace or oven, for drying the dissolvable polymer, polymer resin, or hydrogel. In some examples, the thermal drying device can also provide a negative pressure or vacuum to encourage the polymer resin or hydrogel to dry more quickly. As previously described, the system 210 can also comprise one or more additional centrifuges 224, which can be used for drying the polymer resin and/or hydrogel of assembled hMNAs 10. In some examples, multiple centrifuges can also be used to enable parallel operations to increase the throughput of the manufacturing system.

As previously described, the system 210 can further comprise assembly robots 242, handling robots 252, and/or inspection robots, such as a four-axis or six-axis robotic arms, for moving the molds 110, 150, assembling the MNAs 10, and post-process automated inspection of MNAs, e.g., to satisfy GMP requirements. The assembly robots 242 or handling robots 252 can be configured to assemble different parts of the MNA 10 together either before or after molding and/or curing. For example, the assembly robot 242 or handling robot 252 can mount the adapters 22 over the wells 120 of the micro-cannula mold 110 prior to dispensing the polymer resin to the wells 120. After the adapters 22 are positioned over the wells 120, the conveyors 214a, 214b or other handling robots can move the micro-cannula mold 110 and adapters 22 to the dispensing station. Once the micro-cannula mold 110 is in place, polymer resin can be dispensed through the through-hole 30 of the adapter 22 (e.g., the hole 30 that receives the Luer connector of the syringe) in order to fill or partially fill the wells 120 with polymer resin to make the molded part 12 including the micro-cannula 20 attached to the syringe adapter 22.

With specific reference to the schematic drawing in FIG. 4C, in some examples, the system 210 further comprises a vision system with cameras and/or optical sensors 244 for confirming positioning of the wells 120, 160 of the molds 110, 150. The optical sensor 244 can be a digital camera, such as a 20M pixel digital camera having a resolution better than 10 μm. In some examples, the optical sensor 244 or camera is attached to the assembly robot 242, which can be manipulated to capture images of the molds 110, 150. Information captured by the optical sensor 244 or camera can be used to move and reposition the molds 110, 150 to ensure that the molds 110, 150 are correctly positioned relative to other components of the system 210, such as the dispensers 216a, 216b. The vision system and optical sensors 244 can also be used for assembling different components of the hMNAs 10 together with sufficient precision and accuracy.

The system 210 further comprises one or more controllers 246 electrically connected to components of the system 210 including one or more of the electromechanical movers (e.g., the conveyors 214a, 214b, assembly robots 242, and/or handling robots 252), the dispensers 216a, 216b, centrifuges 224, and/or drying and curing devices 240. The controller 246 can comprise one or more computer processors 248 and system memory 250 comprising instructions that, when executed by the computer processor 248, control dispensing, centrifuging, curing, and drying functions of the system 210 according to a predetermined manufacturing plan. For example, the instructions can cause the processors 248 to automatically process images captured by the optical sensor 244 for alignment, assembly, or inspection. The instructions can also cause the processors 248 to directly communicate with the robots and conveyors of the system 210. In particular, the manufacturing plan can include instructions related to an amount of polymer and resin to dispense into each well 120, 160, a duration of centrifuge cycles for spin-casting the MNA molded parts 12, a duration of curing and/or drying cycles for the MNA molded parts 12, and/or instructions for assembling final MNAs 10 from the MNA molded parts 12.

In some examples, the controller 246 is also in electrical communication with the optical sensor 244 of the camera system. As previously described, information from the optical sensor 244 can be used to correctly position the molds 110, 150 relative to other components of the system 210. In particular, images captured by the optical sensor 244 can be used for aligning the wells 120, 160 of the molds 110, 150 with the nozzle 220 of the dispenser 216a, 216b to ensure that polymer resin is correctly dispersed into each well 120, 160. For example, the controller 246 can be configured to receive images of the molds 110, 150 from the optical sensor 244; analyze the images to identify fiducial markers 132, 172 on the molds 110, 150; and cause one or more of the electromechanical movers, such as the input conveyors 214a, 214b, to move the molds 110, 150 to a position relative to the dispensers 216a, 216b determined based on the analysis of the images. Further, the controller 246 can be configured to prevent the dispensers 216a, 216b from dispensing the polymer resin until the molds 110, 150 are correctly positioned, as determined from the analysis of the images.

In some examples, the controller 246 can also be configured to inspect manufactured MNAs 10 using images captured by cameras or optical sensors 244 of the camera system. For example, the controller 246 can be configured to cause the optical sensor 244 to obtain images of one or more of the assembled MNAs 10 and analyze the obtained images to identify defects in the assembled MNAs 10. The controller 246 can further be configured to cause an electromechanical mover, such as the handling robot 252, to remove MNAs 10 having identified defects from, for example, an output conveyor belt 254 of the system 210.

### Manufacturing Methods

The systems 210 of the present disclosure are configured to perform a number of automated molding, curing, demolding, and assembly steps to make MNAs 10, such as the hMNAs 10 shown in FIGS. 1A and 1B. The following discussion describes molding methods performed by the system 210 for manufacturing hMNAs 10 with assembled adapters 22. As previously described, other types of MNAs 10, such as MNAs 10 having dissolvable microneedles 16, microneedles with through-holes, or coated microneedles, which generally require fewer manufacturing steps, can also be manufactured and assembled using the systems 210 and methods disclosed herein.

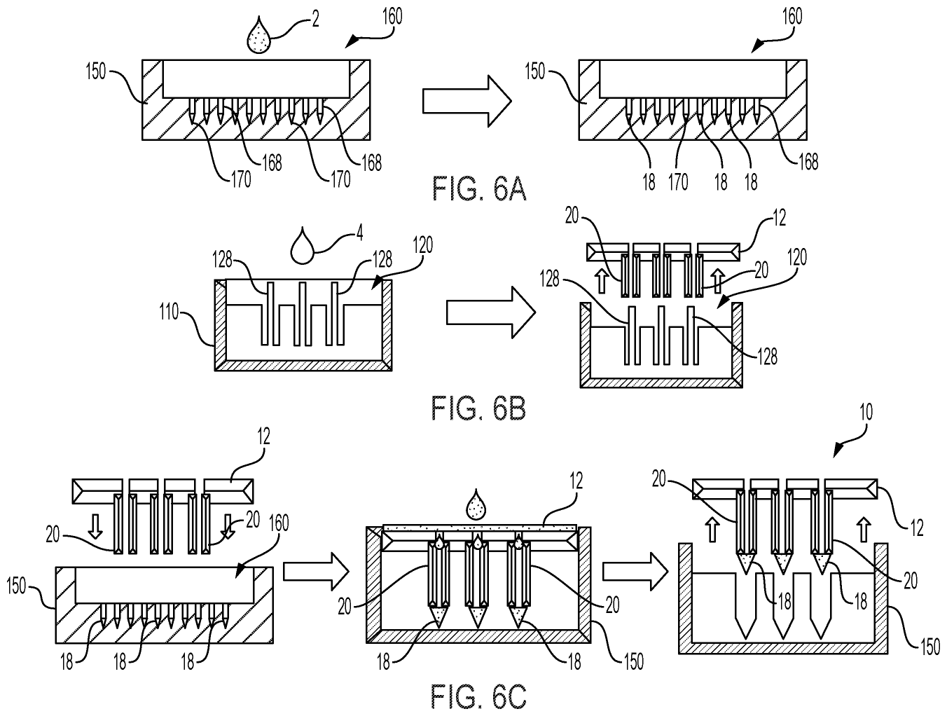

FIGS. 6A-6C are drawings showing molding and assembly steps for making the molded parts 12 and assembled hMNAs 10 of the present disclosure. FIGS. 6A-6C do not show the adapters 22 mounted to the molds 110, 150. However, as previously described, the adapters 22 can be connected to the molds 110, 150 prior to dispensing polymer into the molds 110, 150. More specifically, FIG. 6A shows that dissolvable distal tips 18 can be made by dispensing the hydrogel gel 2 into the tip mold 150, such that the gel 2 collects the tapered distal portion 170 of the holes 168 at the bottom of each well 160. The collected gel 2 is cured to form the distal tips 18. FIG. 6B shows that the molded part 12 including the micro-cannula 20 is made by dispensing the polymer resin 4 to the micro-cannula mold 110 and curing the polymer resin 4 to form the molded part 12. As shown in FIG. 6C, the molded part 12 including the micro-cannula 20 is then inserted into the tip mold 150 including the distal tips 18. Additional hydrogel 2 can be added to the tip mold 150 over the molded part 12 in order to adhere the micro-cannula 20 to the distal tips 18. In some examples, the added hydrogel 2 can also form a base layer or backing 38 of the MNA 10. The tip mold 150 can then be centrifuged and placed in a curing and/or drying device 240 to cure and dry the added hydrogel 2, thereby adhering the molded part 12 to the distal tips 18. After centrifuging and curing, the finished hMNA 10 can be removed from the tip mold 150, as shown in FIG. 6C.

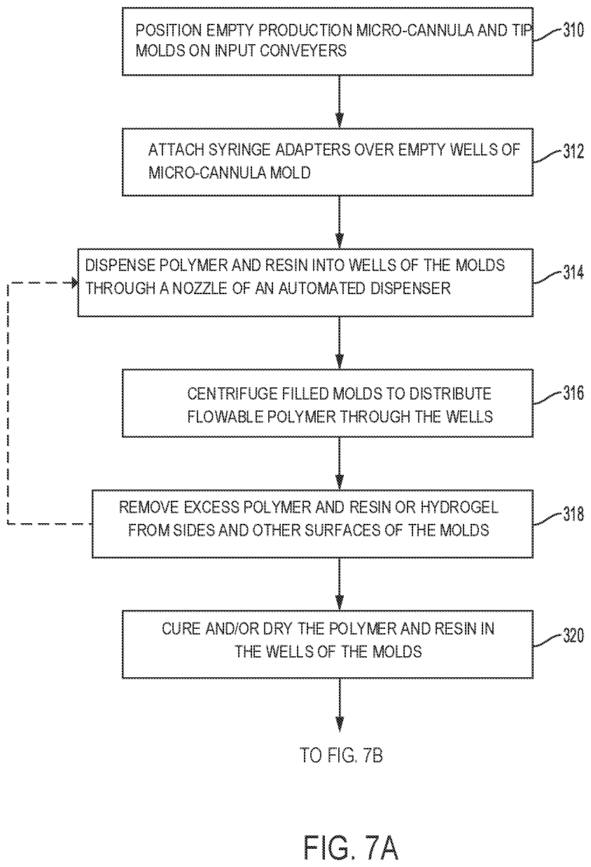

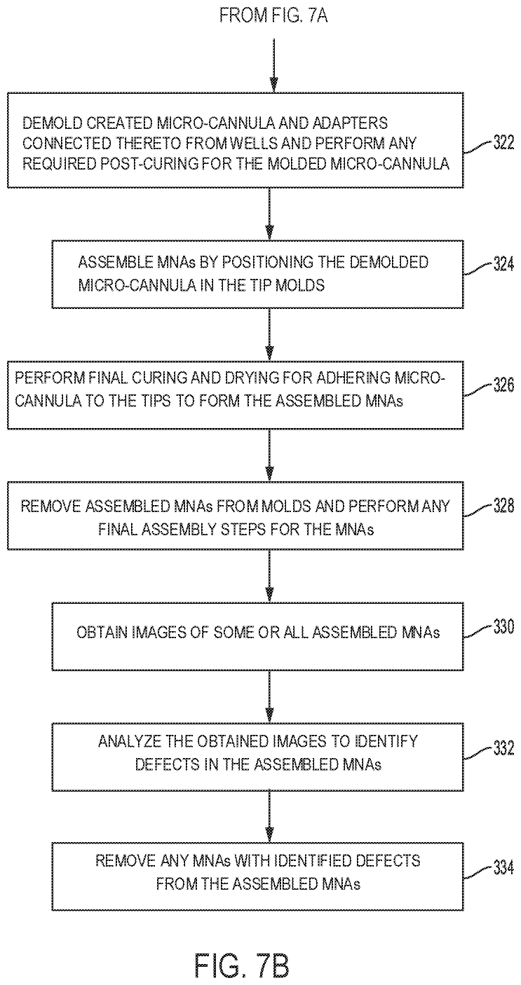

Flow charts showing steps performed by the manufacturing systems 210 of the present disclosure for making the hMNAs 10 are shown in FIGS. 7A and 7B. As shown in FIG. 7A, at step 310, initially, empty production molds (e.g., the micro-cannula mold 110 and the tip mold 150) are placed on the input conveyors 214a, 214b of the system 210. For example, a robotic arm or another electromechanical mover can be used for placing the empty production molds 110, 150 on a base plate 102 and moving the molds 110, 150 and plates 102 to the input conveyors 214a, 214b. Once on the input conveyors 214a, 214b, the molds 110, 150 and plates 102 can be moved to the dispensing station of the system 210.

At step 312, in some examples, the adapters 22, such as syringe adapters 22 made by injection molding or 3D printing, can be positioned over wells 120 of the micro-cannula molds 110 prior to dispensing the polymer resin 4 to the micro-cannula molds 110. For example, an assembly robot 242 or pick and place machine can be used for precisely placing the adapters 22 over the wells 120 as the micro-cannula molds 110 move along the input conveyors 214a, 214b. In other examples, the adapters 22 can be placed over the wells 120 before placing the molds 110 on the conveyors 214a, 214b. As previously described, the adapters 22 can be used for connecting the MNAs 10 to a fluid delivery device, such as a syringe. As previously described, the adapters 22 can comprise a box-shaped body with a through-hole 30 extending from a proximal surface 24 to a distal surface 26 of the adapter 22. The through-hole 30 can be sized to engage a connector, such as a luer slip connector, of the fluid delivery device.

Once the empty molds 110, 150 and/or adapters 22 mounted thereto are in place at the dispensing station, at step 314, the method further comprises dispensing the flowable polymer, such as polymer resin, into wells 120, 160 of the molds 110, 150 by the automated dispensers 216a, 216b comprising the nozzles 220. For example, as previously described, the polymer resin 4 (e.g., a UV curable polymer resin) can be dispensed from the first dispenser 216a, through the through-hole 30 of the adapter 22, to the micro-cannula mold 110. The dissolvable polymer or hydrogel 2 can be dispensed from a second dispenser 216b into the tip mold 150.

At step 316, after dispensing, the filled molds 110, 150 and adapters 22 connected thereto are moved to the centrifuge 224. For example, as previously described, the molds 110, 150 can be placed in the platforms 238a, 238b of the centrifuge bucket 226, which can be mounted in a chamber of the centrifuge 224. Once the molds 110, 150 are in place, the centrifuged 224 can be automatically activated, which distributes the dispensed polymer resin within the wells 120, 160 of the molds 110, 150. Centrifuge cycle times will be determined by those skilled in the art based, for example, on the type of polymer resin 4 dispensed into the wells 120, 160 and/or the dimensions of the molds 110, 150. In some examples, the polymer or hydrogel-loaded tip mold 150 can be centrifuged for about 1 to about 4 minutes to force the hydrogel 2 or another polymer into the holes 168 of the mold 150.

In some examples, the method comprises two centrifugation steps for the tip mold 150 and only one centrifugation step for the micro-cannula mold 110. Specifically, after the hydrogel or other dissolvable polymer is dispensed into wells 160 of the tip mold 150, the filled tip mold 150 is centrifuged for about 7 minutes. This centrifugation step serves to distribute the polymer or hydrogel throughout the wells 170 and fill the tip cavities. This centrifugation step can also “pre-dry” the gel. This centrifugation step can be done at 3500 rpm (2062 xg) and at room temperature (e.g., about 21° C. to about 22° C.), with a tube inserted into the centrifuge chamber that flows and circulates dry air (e.g., air that has passed through desiccant filters) at a flow rate of about 30 L/min. Another tube can be inserted into the centrifuge that also vacuums the air, which serves to speed up the air circulation inside the centrifuge 224 and suck evaporated water from the hydrogel. The applied vacuum speeds up the drying process.

As described in further detail hereinafter, additional centrifugation steps for the tip mold 150 can be performed later in the manufacturing method. For example, once this first centrifugation step is completed, the fully formed hollow MNA parts 12 with the adaptors 22 attached thereto can be removed from the micro-cannula mold 110 and placed into the filled tip mold 150 by robotic arms of the system 210. Then, additional centrifugation steps can be performed. For example, the mold 150 with the gel forming the tip cavities and the previously formed micro-cannula and adaptors 10 can centrifuged in a second or subsequent centrifugation step for about 1.5 hours, with the same settings and attachments as listed previously. This second or subsequent centrifugation step serves to create the MNAs 10 with the dried dissolvable tips.

For the micro-cannula molds 110, after the polymer resin is dispensed, the molds 110 are centrifuged to distribute the resin. The settings for this centrifugation step can be about 2000 rpm (850 xg) to about 3500 rpm (2602 xg). The centrifuge can be activated for only a short period, such as about 1 minute. The temperature is again about room temperature (e.g., about 21° C. to about 22° C. The previously described dry air input and vacuum can still be present during this step. However, the input air and vacuum may not be needed because polymer resin does not contain water content that needs to be evaporated.

With continued reference to FIG. 7A, after centrifuging, the electromechanical movers, such as a handling robot 252, can be configured to remove the molds 110, 150 from the centrifuge 224. At step 318, the assembly and/or handling robots 242, 252 can also be configured to scrape off excess polymer resin 4 or hydrogel 2 from sides and other surfaces of the molds 110, 150 using a scraper. For example, the scraper can be a sharp-edged knife with 8-12 edges configured to simultaneously scrape multiple wells 120, 160 of the molds 110, 150. In other examples, as previously described, a “pillared push plate” can be used for distributing the polymer resin 4 or hydrogel 2 through the wells 120, 160 of the molds 110, 150. The “pillars” of the “pillared push plate” can be fabricated such that the pillars “push” over surfaces of the molds 110, 150 at, for example, locations between openings of the microneedle holes 168 of the tip mold 150.

After the push plate is placed onto the molds 110, 150, the robots 242, 252 can place the molds 110, 150 and push plate mounted thereto back into the centrifuge 224 for another centrifugation cycle (e.g., 1-2 minute centrifugation at >2,000 xg). This pillared push plate can be configured to both distribute the hydrogel 2 or another polymer through the wells 120, 160 and to remove excess polymer or hydrogel in one step (through capillary action, where the excess polymer or hydrogel 2 between the pillars is removed with the push plate).

In another example, a channeling plate, which is essentially an inverse of the push plate, can be used for distributing the polymer through the molds 110, 115. Specifically, the channeling plate can comprise holes that align with holes the well of the tip mold 115, but which have concavities around each hole. The channeling plate can be placed on the tip mold 115 before the gel or another polymer is dispensed onto the mold 115. The concavities around the holes of the channeling plate can help direct polymer to flow into the tip holes of the mold below. This arrangement can be used to minimize hydrogel waste, meaning that less gel can be dispensed onto the mold 115.

After these centrifugation steps, one of the assembly robots 242 or handling robots 252 can remove the molds 110, 150 from the centrifuge 224 and place the centrifuged molds 110, 150 onto a handling platform. The handling robot 252 can then remove the pillared push plates from the molds 110, 150. The pillared push plates can be cleaned and reused. In some examples, an additional centrifuging process can be performed at this stage for the tip mold 150 to increase the concentration of the hydrogel 2 or another polymer in the microneedles 16.

The molds 110, 150 next can be moved to the curing and/or drying device 240, such as the previously-described UV curing cabinet, for exposing the curable polymer resin 4 to UV radiation. In some examples, the UV cabinet can accommodate multiple molds 110, 150, such as four, six, eight, or more molds 110, 150. In some examples, it may take about 5 minutes to fully cure the polymer resin 4. Once in place in the curing and/or drying device 240, at step 320, the curing and/or drying device 240 can be activated for curing and/or drying the polymer resin within the wells 120, 160 of the molds 110, 150. In some examples, the dispensing, centrifuging, and curing/drying steps can be performed multiple times in order to form a molded part 12 comprising several separately formed layers.

With reference to FIG. 7B, after curing and drying, at step 322, demolding and post-curing can be performed. In some examples, demolding of the molded parts 12 from the wells 120, 160 is performed by the assembly robots 242 or handling robots 252. For example, some or all of the molded parts 12 comprising the micro-cannula 20 and adapters 22 mounted thereto can be removed from the micro-cannula molds 110. The demolded parts 12 can be further post-cured to ensure the full curing of any residual resin and to further increase mechanical strength of the molded parts 12.

When the resin 4 post-curing is complete, the molded part 12 includes fully formed micro-cannulas 20. Also, the adapters 22 are well-adhered to the molded part 12. In particular, due to the design of the adapters 22 and wells 120, 160, filling resin into the wells 120, 160, enables attachment of a backing 38 to the sides of the adapters 22, which secures the adapters 22 to the molded parts 12. In some examples, the adapters 22 can be further surface-treated to facilitate adhesion to the polymer material of the molded part 12. The material selection (both for the adapter 22 and the molded part 12) and the geometric design of the adapter 22 and molded part 12 can also be adapted for precisely and strongly adhering the adapter 22 and molded part 12 together.

At step 324, after demolding and post-curing, an assembly step can be performed where the molded parts 12 and adapters 22 connected thereto are positioned over the wells 120, 160 of the tip mold 150 (as shown in FIG. 6C). Once the molded parts 12 are in position, an additional amount of hydrogel 2 or another polymer can be deposited over the molded part 12, thereby adhering the molded part 12 to the dissolvable distal tips 18 comprising the hydrogel 2 formed in the tip mold 150.

In some examples, the process of placing the molded part 12 and adapters 22 connected thereto onto the wells 120, 160 of the tip mold 150 may need to be performed with high precision and accuracy, such as better than 20 μm accuracy, to ensure that each micro-cannula 16 of the molded part 12 is placed into a microneedle mold hole 168 of the tip mold 150. Further, the adapters 22 may be manufactured with better than 10 μm accuracy to ensure the adapters 22 can be precisely positioned relative to the wells 160. In some examples, as previously described, the optical sensor 244 of the system 210 is used to determine the location of the wells 160 and holes 168 of the tip mold 150 to provide the required precise positioning. In particular, the robots 242, 252 can be configured to pick up each adapter 22 and place it accurately and precisely in its respective position on the hydrogel-filled tip mold 150 at positions determined from analysis of images captured by the optical sensors 244 or cameras. Only a mild pressure is used to insert the adapters 22 and molded parts 12 connected thereto in place on the wells 160 of the tip mold 150.

At step 326, after the additional amount of hydrogel 2 or another polymer is dispensed for securing the molded part 12 to the dissolvable tips distal 18, the tip mold 150 and adapter 22 can be moved to the curing and/or drying device 240 for final curing and drying of the hydrogel 2 or another polymer. For example, as previously described, the “assembled” hMNAs 10 in the tip molds 150 can be placed back into the centrifuge 224, which can be adapted to accommodate from four to eight tip molds 150 at a time. The tip molds 150 are then centrifuged for a long duration, such as from about 15 minutes to about 75 minutes until the hydrogel 2 or another polymer is fully dry. Warm and/or dry air can be applied to the centrifuge 224 to cause the hydrogel 2 or other dissolvable polymer material to dry more quickly. When the hydrogel 2 or another polymer is fully dry, the tips 18 are fully formed and are securely attached to the micro-cannulas 20 of the molded part 12. Once the hydrogel 2 or another polymer is dry, the hMNAs 10 can be removed from the tip mold 150 using, for example, one or more electromechanical movers for grasping and removing the hMNA 10 from the tip mold 150.

At step 328, after removal from the tip mold 150, any final assembly steps can be performed to form the finalized hMNAs 10. For example, if not already present, adapters, backing layers, or similar structures can be added to the molded hMNAs 10. In some examples, portions of the hMNAs 10 can be coated with, for example, therapeutic agents or materials to improve adhesive between the hMNAs 10 and the patient's skin. Following final assembly, the completed hMNAs 10 can be positioned on a mover, such as on an output conveyer 254, for moving the completed hMNAs 10 from the manufacturing unit or cell to a final collection, packaging, or distribution facility.

At step 330, in some examples, the method can further comprise obtaining images for some or all of the assembled hMNAs 10 with the optical sensors 244 or cameras. For example, the optical sensors 244 or cameras can be positioned to capture images of the assembled hMNAs 10 moving along the output conveyor belt 254. In some examples, the system 210 is configured to obtain images of from about 5% to about 20% of the assembled hMNAs 10 for inspection purposes.

At step 332, the method can further comprise analyzing the obtained images with the controller 246 or computer processor to identify defects in the assembled hMNAs 10. For example, a defect can include an hMNA 10, wherein the adapter 22 is not correctly aligned relative to other molded MNA parts. Defects may also include molding defects, such as MNAs 10 having fewer microneedles 16 than expected or hMNAs 10 having incomplete microneedles 16.

At step 334, the method can further comprise removing any hMNAs 10 with identified defects from the assembled hMNAs 10. For example, another electromechanical mover, such as a pick and place machine or automated robotic arm, can be used for removing hMNAs 10 with defects from the output conveyor 254. The removed hMNAs 10 can be discarded.

Non-limiting aspects or embodiments of the present invention will now be described in the following numbered clauses:

Clause 1: A manufacturing method for making a plurality of microneedle arrays (MNAs), the method comprising: dispensing a polymer resin into a plurality of wells of at least one mold by an automated dispenser comprising at least one dispensing nozzle; centrifuging the at least one mold to distribute the dispensed polymer resin within the plurality of wells of the at least one mold; curing, solidifying, and/or drying the polymer resin within the plurality of wells of the at least one mold; and removing individual molded MNA parts from the at least one mold with at least one electromechanical mover controlled by at least one computer processor.

Clause 2: The method of clause 1, further comprising assembling the plurality of MNA from the molded MNA parts with the at least one electromechanical mover.

Clause 3: The method of clause 1 or clause 2, wherein the at least one mold comprises: a tray comprising a top surface, a bottom surface, and a sidewall extending between the top surface and the bottom surface; and the plurality of the wells on the top surface of the tray, each well comprising an open top, a closed bottom, and an inner surface extending between the open top and the closed bottom.

Clause 4: The method of clause 3, wherein the at least one mold further comprises a plurality of fiducial markers on the top surface of the tray, and wherein dispensing the polymer resin to the plurality of wells comprises obtaining images of the at least one mold with an optical sensor, analyzing the obtained images with the at least one computer processor to identify at least one of the plurality of fiducial markers in the images, and aligning the at least one mold with the automated dispenser based on a position of the at least one identified of the plurality fiducial marker in the analyzed images.

Clause 5: The method of clause 3 or clause 4, wherein the tray comprises 96 wells and corresponds in size (e.g., length and width) to a standard 96 well tray used for biological sampling.

Clause 6: The method of any of clauses 1 to 5, wherein the at least one mold further comprises at least one hole on the closed bottom of the plurality of wells for forming microneedles of the MNAs.

Clause 7: The method of clause 6, wherein a bottom portion of the at least one hole is tapered for forming a sharpened needle tip of the microneedles.

Clause 8: The method of any of clauses 1 to 5, wherein the at least one mold further comprises at least one post extending from the closed bottom of the plurality of wells towards the open top of the plurality of wells for forming micro-cannula of the MNAs.

Clause 9: The method of any of clauses 2 to 5, wherein the at least one mold comprises a micro-cannula mold and a tip mold, and wherein dispensing the polymer resin material comprises dispensing UV curable polymer resin to the micro-cannula mold and dispensing a polymer to the tip mold.

Clause 10: The method of clause 9, wherein assembling the plurality of MNAs comprises inserting molded MNA parts formed in the micro-cannula mold into the plurality of wells of the tip mold, after the polymer is dispersed to the tip mold, to form MNAs comprising a plurality of micro-cannula covered by distal tips comprising the polymer.

Clause 11: The method of any of clauses 1-10, wherein the centrifuge comprises a support contained within a bucket of the centrifuge for containing multiple molds in a stacked configuration, and wherein centrifuging the at least one mold comprises, after filling the at least one mold, placing the at least one mold on an upper platform of the support, placing another filled mold on a lower platform of the support, and activating the centrifuge to distribute the polymer resin through the plurality of wells of the filled molds.

Clause 12: The method of any of clauses 1-11, wherein the polymer resin is dispensed into a well of the plurality of wells through a multi-head nozzle having two, three, four, or more heads configured to distribute the polymer resin throughout the well of the plurality of wells.

Clause 13: The method of any of clauses 1-12, wherein the dispensing, centrifuging, and curing steps are repeated multiple times to provide a multi-layer MNA.

Clause 14: The method of any of clauses 1-13, further comprising obtaining images of the assembled MNAs with at least one optical sensor and analyzing the obtained images with the at least one computer processor to identify defects in the assembled MNAs.

Clause 15: The method of clause 14, further comprising removing any MNAs with identified defects from a group of assembled MNAs.

Clause 16: The method of clause 15, wherein the images are obtained and analyzed for from about 5% to about 20% of the MNAs.

Clause 17: The method of clause 2, further comprising forming the adapters by at least one of injection molding or 3D printing, and wherein assembling the plurality of MNAs comprises attaching an adapter to a top surface of the MNA parts with the at least one electromechanical mover.

Clause 18: The method of clause 17, wherein the adapters comprise a body and at least one tapered through-hole configured to receive a male Luer connector (e.g., a male Luer slip connector or a male Luer lock connector) of a fluid delivery device.

Clause 19: The method of any of clauses 1 to 17, wherein the at least one mold is an elastomeric production mold, the method further comprising: forming the production mold in a rigid master mold by dispensing polymer resin into the master mold and curing the polymer resin to form the production mold, and demolding the formed production mold from the master mold by placing the master mold in a centrifuge in an upside down configuration and activating the centrifuge to cause the production mold to release from the master mold.

Clause 20: A mold for making a plurality of microneedle arrays (MNAs), comprising: a tray comprising a top surface, a bottom surface, and a sidewall extending between the top surface and the bottom surface; a plurality of wells on the top surface of the tray, each well comprising an open top, a closed bottom, and an inner surface extending between the open top and the closed bottom; and a plurality of fiducial markers on the tray that can be identified in images of the mold captured by optical sensors for aligning the mold with a dispenser of an MNA manufacturing system.

Clause 21: The mold of clause 20, wherein the tray comprises 96 wells and corresponds in size (e.g., length and width) to a standard 96 well tray used for biological sampling.

Clause 22: The mold of clause 20, wherein the tray comprises either 8 wells or 24 wells and corresponds in size to a standard tray used for biological sampling.

Clause 23: The mold of any of clauses 20-22, wherein the tray comprises an elastomer, such as siloxanes (e.g., polydimethylsiloxane (PDMS)), a thermoplastic, or a light-curable resin.

Clause 24: The mold of any of clauses 20-23, further comprising a super-hydrophobic coating over surfaces of the closed bottom and the inner surface of the plurality of wells.

Clause 25: The mold of clause 24, wherein the super-hydrophobic coating comprises at least one biocompatible polymer with low surface energy, such as fluorinated chlorosilanes, such as Trichloro(1H,1H,2H,2H-perfluorooctyl)silane (PFOCTS).

Clause 26: The mold of any of clauses 20 to 25, further comprising at least one hole on the closed bottom of the plurality of wells for forming microneedles of the plurality of MNAs.

Clause 27: The mold of clause 26, wherein the at least one hole has a square or a rectangular cross-section.

Clause 28: The mold of clause 26 or clause 27, wherein the mold comprises a plurality of holes on the closed bottom of each well arranged in rows and columns, with a hole being aligned with an adjacent hole in both a width and length direction, or wherein the plurality of holes are arranged in a concentric circular pattern.

Clause 29: The mold of any of clauses 26 to 28, wherein a bottom of the at least one hole is tapered for forming sharpened needle tips in the plurality of holes.

Clause 30: The mold of clause 26, wherein each well comprises from 1 hole to about 1000 holes.

Clause 31: The mold of any of clauses 20 to 25, further comprising at least one post extending from the closed bottom of the plurality of wells towards the open top of the plurality of wells.

Clause 32: The mold of clause 31, wherein the at least one post has a circular cross section.

Clause 33: The mold of clause 31 or clause 32, wherein the mold comprises a plurality of posts in each well, and wherein the plurality of posts are arranged in rows and columns, with a post being aligned with an adjacent post in both a width and length direction, or wherein the plurality of posts are arranged in a concentric circular pattern.

Clause 34: The mold of clause 31, wherein each well comprises from 1 post to about 1000 posts.

Clause 35: A high-throughput manufacturing system for making microneedle arrays (MNAs), the system comprising: at least one mold for making a plurality of microneedle arrays (MNAs); at least one dispenser that dispenses a polymer resin material for forming a molded MNA part to a plurality of wells of the at least one mold; at least one centrifuge for centrifuging the at least one mold causing the polymer resin material dispensed into the plurality of wells of the at least one mold to distribute through the plurality of wells; at least one curing or drying device for exposing the filled at least one mold to heat and/or radiation causing the polymer resin material to cure or dry; and at least one automated or robotic electromechanical mover for moving the at least one mold through the at least one dispenser, the at least one centrifuge, and the at least one curing or drying device, for demolding the molded MNA parts from the at least one mold, and for assembling the MNAs from the molded MNA parts.

Clause 36: The system of clause 35, wherein the at least one dispenser comprises at least one nozzle for dispensing the polymer resin to the plurality of wells, wherein the at least one nozzle comprises a multi-head nozzle comprising two, three, four, or more heads for dispersing multiple fluid streams to the plurality of wells.

Clause 37: The system of clause 35 or clause 36, wherein the at least one dispenser comprises a first dispenser for dispensing a solution comprising a dissolved gel (hydrogel) and a second dispenser for dispensing the polymer resin.

Clause 38: The system of any of clauses 35 to 37, wherein the at least one mold comprises a micro-cannula mold for forming a micro-cannula of the MNA parts and a tip mold for forming dissolvable hydrogel tips of the MNA parts.

Clause 39: The system of any of clauses 35-38, wherein the at least one automated or robotic electromechanical mover comprises a linear actuator, a conveyor, a 4-axis robotic arm, a 6-axis robotic arm, a mechanical lift, and/or a pick and place machine.

Clause 40: The system of any of clauses 35-39, wherein the at least one automated or robotic electromechanical mover comprises at least one robotic arm configured to place an adapter over the plurality of wells of the at least one mold.

Clause 41: The system of clause 40, wherein the at least one dispenser dispenses the polymer resin into the plurality of wells through a through-hole extending through the adapter.

Clause 42: The system of clause 41, wherein the through-hole of the adapter is tapered for receiving a Luer connector (e.g., a Luer slip connector or a Luer Lock connector) of a fluid delivery device.

Clause 43: The system of clause 42, wherein the at least one centrifuge comprises a bucket comprising a platform positioned in the bucket with the at least one mold mounted to the platform.

Clause 44: The system of clause 43, wherein the platform comprises an upper platform and a lower platform, with each platform receiving the at least one mold.

Clause 45: The system of any of clauses 35-44, wherein the at least one curing or drying device comprises a container for receiving the at least one mold, the curing or drying device configured to expose the at least one mold to ultraviolet radiation, heated air, and/or a vacuum.

Clause 46: The system of clause 45, wherein the at least one curing or drying device further comprises an additional centrifuge for rotating the at least one mold to dry the polymer resin.

Clause 47: The system of any of clauses 35-46, wherein the at least one automated or robotic electromechanical mover comprises at least two input conveyors for moving a first mold and a second mold to the at least one dispenser, at least one handling robot for moving the first mold and/or the second mold to the at least one centrifuge and/or the at least one curing and/or drying device, at least one assembly robot for demolding MNA parts from the first mold and/or the second mold and for assembling the MNAs from the demolded MNA parts, and at least one output conveyor for conveying the assembled MNA parts away from the at least one assembly robot.

Clause 48: The system of any of clauses 35-47, further comprising at least one computer processor in electronic communication with the at least one dispenser, the at least one centrifuge, the at least one curing and/or drying device, and/or the at least one automated or robotic electromechanical mover, wherein the at least one computer processor is configured to control dispensing, centrifuging, curing, or drying of the at least one mold according to a predetermined manufacturing plan.

Clause 49: The system of clause 48, wherein the at least one computer processor is further configured to cause the at least one automated or robotic electromechanical mover to move the at least one mold from the at least one dispenser, to the at least one centrifuge, and to the at least one curing or drying device according to the predetermined manufacturing plan.

Clause 50: The system of clause 48 or clause 49, further comprising at least one optical sensor in electronic communication with the at least one computer processor for obtaining images of the at least one mold, wherein the at least one computer processor is configured to: receive the images of the at least one mold from the at least one optical sensor; analyze the images to identify at least one fiducial marker on the at least one mold; and cause the at least one automated or robotic electromechanical mover to move the at least one mold to a position relative to the at least one dispenser determined based on the analysis of the images.

Clause 51: The system of clause 50, wherein the at least one computer processor is configured to prevent the at least one dispenser from dispensing the polymer resin until the at least one mold is correctly positioned, as determined from the analysis of the images.

Clause 52: The system of clause 50 or clause 51, wherein the at least one computer processor is further configured to cause the at least one optical sensor to obtain the images of one or more of the assembled MNAs and analyze the obtained images to identify defects in the assembled MNAs.

Clause 53: The system of clause 52, wherein the at least one computer processor is configured to cause the at least one automated or robotic electromechanical mover to remove the MNAs having identified defects.

1. A manufacturing method for making a plurality of microneedle arrays (MNAs), the method comprising:
dispensing a polymer resin into a plurality of wells of at least one mold by an automated dispenser comprising at least one dispensing nozzle;
centrifuging the at least one mold to distribute the dispensed polymer resin within the plurality of wells of the at least one mold;
curing, solidifying, and/or drying the polymer resin within the plurality of wells of the at least one mold; and
removing individual molded MNA parts from the at least one mold with at least one electromechanical mover controlled by at least one computer processor.

2. The method of claim 1, further comprising assembling the plurality of MNA from the molded MNA parts with the at least one electromechanical mover.

3. The method of claim 1, wherein the at least one mold comprises:
a tray comprising a top surface, a bottom surface, and a sidewall extending between the top surface and the bottom surface; and
the plurality of wells on the top surface of the tray, each well comprising an open top, a closed bottom, and an inner surface extending between the open top and the closed bottom.

4. The method of claim 3, wherein the at least one mold further comprises a plurality of fiducial markers on the top surface of the tray, and wherein dispensing the polymer resin to the plurality of wells comprises obtaining images of the at least one mold with an optical sensor, analyzing the obtained images with the at least one computer processor to identify at least one of the plurality of fiducial markers in the images, and aligning the at least one mold with the automated dispenser based on a position of the at least one of the plurality of identified fiducial marker in the analyzed images.

5. The method of claim 3, wherein the tray comprises 96 wells and corresponds in size (e.g., length and width) to a standard 96 well tray used for biological sampling.

6. The method of claim 1, wherein the at least one mold further comprises at least one hole on the closed bottom of the plurality of wells for forming microneedles of the MNAs.

7. The method of claim 6, wherein a bottom portion of the at least one hole is tapered for forming a sharpened needle tip of the microneedles.

8. The method of claim 1, wherein the at least one mold further comprises at least one post extending from the closed bottom of the plurality of wells towards the open top of the plurality of wells for forming micro-cannula of the MNAs.

9. The method of any of claim 1, wherein the at least one mold comprises a micro-cannula mold and a tip mold, and wherein dispensing the polymer resin material comprises dispensing UV curable polymer resin to the micro-cannula mold and dispensing a polymer to the tip mold.

10. The method of claim 9, wherein assembling the plurality of MNAs comprises inserting molded MNA parts formed in the micro-cannula mold into the plurality of wells of the tip mold, after the polymer is dispersed to the tip mold, to form MNAs comprising a plurality of micro-cannula covered by distal tips comprising the polymer.

11. The method of claim 1, wherein the centrifuge comprises a support contained within a bucket of the centrifuge for containing multiple molds in a stacked configuration, and wherein centrifuging the at least one mold comprises, after filling the at least one mold, placing the at least one mold on an upper platform of the support, placing another filled mold on a lower platform of the support, and activating the centrifuge to distribute the polymer resin through the plurality of wells of the filled molds.

12. The method of claim 1, wherein the polymer resin is dispensed into a well of the plurality of wells through a multi-head nozzle having two, three, four, or more heads configured to distribute the polymer resin throughout the well of the plurality of wells.

13. The method of claim 1, wherein the dispensing, centrifuging, and curing steps are repeated multiple times to provide a multi-layer MNA.

14. The method of claim 1, further comprising obtaining images of assembled MNAs with at least one optical sensor and analyzing the obtained images with the at least one computer processor to identify defects in the assembled MNAs.

15. The method of claim 14, further comprising removing any MNAs with identified defects from a group of assembled MNAs.

16. (canceled)

17. The method of claim 2, further comprising forming the adapters by at least one of injection molding or 3D printing, and
wherein assembling the plurality of MNAs comprises attaching an adapter to a top surface of the MNA parts with the at least one electromechanical mover.

18. The method of claim 17, wherein the adapters comprise a body and at least one tapered through-hole configured to receive a male Luer connector (e.g., a male Luer slip connector or a male Luer lock connector) of a fluid delivery device.

19. The method of claim of 1, wherein the at least one mold is an elastomeric production mold, the method further comprising:
forming the production mold in a rigid master mold by dispensing polymer resin into the master mold and curing the polymer resin to form the production mold, and
demolding the formed production mold from the master mold by placing the master mold in a centrifuge in an upside down configuration and activating the centrifuge to cause the production mold to release from the master mold.

20. A mold for making a plurality of microneedle arrays (MNAs), comprising:
a tray comprising a top surface, a bottom surface, and a sidewall extending between the top surface and the bottom surface;
a plurality of wells on the top surface of the tray, each well comprising an open top, a closed bottom, and an inner surface extending between the open top and the closed bottom; and
a plurality of fiducial markers on the tray that can be identified in images of the mold captured by optical sensors for aligning the mold with a dispenser of an MNA manufacturing system.

21-34. (canceled)

35. A high-throughput manufacturing system for making microneedle arrays (MNAs), the system comprising:
at least one mold for making a plurality of MNAs;
at least one dispenser that dispenses a polymer resin material for forming a molded MNA part to a plurality of wells of the at least one mold;
at least one centrifuge for centrifuging the at least one mold causing the polymer resin material dispensed into the plurality of wells of the at least one mold to distribute through the plurality of wells;
at least one curing or drying device for exposing the filled at least one mold to heat and/or radiation causing the polymer resin material to cure or dry; and
at least one automated or robotic electromechanical mover for moving the at least one mold through the at least one dispenser, the at least one centrifuge, and the at least one curing or drying device, for demolding the molded MNA parts from the at least one mold, and for assembling the MNAs from the molded MNA parts.

36-53. (canceled)
